(19) United States
(12) Patent Application Publication
(10) Pub. No.: US 2023/0391821 A1
(43) Pub. Date: Dec. 7, 2023

(54) METHODS FOR TARGET RELEASE FROM INTEIN COMPLEXES

(71) Applicant: MERCK PATENT GMBH, DARMSTADT (DE)

(72) Inventors: Oliver RAMMO, Darmstadt (DE); Vanessa KOHL, Darmstadt (DE)

(73) Assignee: MERCK PATENT GMBH, DARMSTADT (DE)

(21) Appl. No.: 18/028,376

(22) PCT Filed: Sep. 27, 2021
(86) PCT No.: PCT/EP2021/076448

§ 371 Date: Mar. 24, 2023

(30) Foreign Application Priority Data
Sep. 28, 2020 (EP) 20198728.6

(51) Int. Cl. C07K 1/22; C12P 21/06; C12N 15/62; C07K 14/245

(52) U.S. Cl. CPC C07K 1/22; C12P 21/06; C07K 2319/22; C07K 14/245; C07K 2319/92; C12N 15/62

(57) ABSTRACT

The present disclosure relates to methods of releasing a target molecule from intein complexes comprising an intein-C tagged target molecule and intein-N polypeptides, by contacting the intein complexes with nitrogen containing heteroaromatic derivatives, and/or by increasing residence time of intein complexes in a medium effective to remove the target molecule. Modulating the pH further facilitates target release.

### FIELD

The present disclosure relates to methods of releasing a target molecule from intein complexes comprising an intein-C tagged target molecule and intein-N polypeptides, by contacting the intein complexes with nitrogen containing heteroaromatic compounds, and/or by increasing residence time of intein complexes in a medium effective to remove the target molecule. Modulating the pH further facilitates target release.

### BACKGROUND

Inteins are autocatalytic proteins that are capable of self-splicing from a precursor protein, resulting in a joining of the flanking proteins (exteins) via a peptidic bond. Inteins have become increasingly popular for diverse applications in biotechnology, chemical biology and synthetic biology, because of their ability to tolerate deliberate exchange of extein sequences, as well as the existence of naturally occurring split inteins reconstituting a functional protein from two polypeptide chains.

In one application, intein technology can be used for purification of target proteins. The intein specific splicing process can be modified through various mutations including a single point mutation in the N-terminal intein fragment to result in only C-terminal splicing activity, i.e., Cleavage. Thus, intein-N fragments can be immobilized as affinity ligand on a chromatographic support whereas the intein-C serves as purification tag on the target molecule, e.g., a protein. Due to their ability to specifically associate under given conditions, the intein-C tagged target molecule can be successfully isolated from a feed stock whereas all the other impurities stay in the flow-through. The release of the target protein is subsequentially induced through a change in the buffer system driven by additives such as thiol containing compounds or reducing agents, pH or temperature.

However, there remains a need to control the capture and cleavage reaction so as to achieve complete release of the tagless target molecule at high splicing rates, with no premature cleavage at the level of intein-C tagged target capture. It has been shown that premature splicing and cleavage activity can be successfully suppressed by the addition of divalent ions such as Zinc. In a subsequent step, the ions are removed through the addition of chelating agents such as EDTA or thiol containing compounds such as DTT (Guan et el. 2013, Biotechnol Bioeng. 110(9):2471-81), triggering the target release. Alternatively, the cleavage reaction can also be controlled through the combination of temperature and pH shift (Belfort et al. U.S. Pat. No. 6,933,362, Lu et. al. 2011, J Chromatogr A. 1218(18):2553-60). However, all conditions and methods reported to date are not suitable for a large-scale chromatographic purification process. Thus, there is an unmet need in the art for efficient methods to control target release from intein columns, which provides efficient release of the target molecule and minimizes or avoids premature release.

### SUMMARY

The present disclosure relates to methods of releasing a target molecule from intein complexes comprising an intein-C tagged target molecule and intein-N polypeptides, by contacting the intein complexes with nitrogen containing heteroaromatic compounds, and/or by increasing residence time of intein complexes in solutions effective to remove the target molecule. Modulating the pH of a solution comprising the intein complex further facilitates target release.

It has now surprisingly been found that target release from intein complexes comprising a covalently-bound target molecule (e.g., proteins) can be controlled by addition of nitrogen-containing heteroaromatic compounds such as azoles (e.g., imidazole, pyrazole, oxazole) and azole-containing compounds (e.g., histidine). Additionally and alternatively, it has now been discovered that longer residence time of an intein complex in a solution effective to release the target molecule, as compared with the residence time utilized to form the intein complex, facilitates target cleavage. Reduction of the pH of the solution during target removal further enhances release of the target molecule. With these manipulations, the enzymatic release reactions can be controlled leading to faster splicing/cleavage rates and a higher yield of target release.

Thus, in one embodiment, the present disclosure provides a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of: (a) contacting the intein-C tagged target molecule with the intein-N polypeptide, for a first time period sufficient to form the intein complex; and (b) releasing the target molecule from the intein complex by: (i) contacting the intein complex with a medium effective to remove the target molecule, for a second time period which is longer than the first time period; and/or (ii) contacting the intein complex with a heteroaromatic compound comprising at least one ring nitrogen atom.

In another embodiment, the present disclosure provides a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of contacting the intein complex with a heteroaromatic compound comprising at least one ring nitrogen atom.

The present disclosure exemplifies this concept with intein-mediated protein purification processes, whereby intein complexes are formed on a chromatography resin and the target molecule is cleaved and eluted by decreasing the column flow rate (leading to a higher column residence time), and/or adding nitrogen-containing compounds (e.g., heteroaromatics) and further optionally modulating the pH. As demonstrated herein, the methods may be consistently applied throughout different purification processes involving intein-N fragments immobilized on a chromatographic support.

While the methods of the present disclosure have been exemplified for protein purification processes, it is apparent to a person of skill in the art that the methods of the disclosure are applicable to a variety of intein-mediated processes including but not limited protein ligation, in vivo protein tagging, protein labelling, protein cyclization, protein polymerization, intein-induced reporter pathway analysis, and preparation of fusion proteins. Additionally, due to the structural and functional homology in intein fragments, it is apparent to a person of skill in the art that the methods described herein are applicable for a wide variety of intein-C and intein-N complexes.

### BRIEF DESCRIPTION OF THE DRAWINGS

The disclosure is illustrated in the figures of the accompanying drawings which are meant to be exemplary and not limiting, in which like references are intended to refer to like or corresponding parts, and in which:

FIG. 1A-B depicts A280-Absorbance chromatogram overlays of 3 intein purification cycles using different Cleavage Buffers B.1-B.3. FIG. 1A: intein-N ligand prototype column carrying R44-358132, using a 13 kDa intein-C tagged target (Example 2-1). FIG. 1B: intein-N ligand prototype column carrying R46-358232, using a 36 kDa intein-C tagged target (Example 2-2). Columns were loaded 3 times with pre-purified intein-C tagged target solution. The target was eluted using different Cleavage Buffers: B 1: reference cycle using standard Cleavage Buffer (100 mM Tris, 200 mM NaCl, pH 7) without additives. B2: Cleavage Buffer+0.3 M imidazole. B3: Cleavage Buffer+0.6 M imidazole.

FIG. 2 depicts eluted target amount during five intein purification cycles using Cleavage Buffers B.1-B.5 using a 13 kDa intein-C tagged target (FIG. 2A: Example 2-1); or 36 kDa intein-C tagged target (FIG. 2B: Example 2-2). 1: Cleavage Buffer. B2: Cleavage Buffer+0.3 M imidazole. B3: Cleavage Buffer+0.6 M imidazole. B4: Cleavage Buffer+0.3 M histidine. B5: Cleavage Buffer+0.3 M pyrazole.

FIG. 3A-B: SDS Page analysis showing protein composition of the elution step 1 (E1) and elution step 2 (E2) of Examples 2-1 (FIG. 3A) and 2-2 (FIG. 3B). E1: Elution fraction 1. E2: Elusion fraction 2. B 1: Cleavage Buffer. B2: Cleavage Buffer+0.3 M imidazole. B3: Cleavage Buffer+0.6 M imidazole. B4: Cleavage Buffer+0.3 M histidine. B5: Cleavage Buffer+0.3 M pyrazole.

FIG. 4A-D: Overlay of the elution fractions of Example 2, analyzed by microfluidic electrophorese system. FIG. 4A, 4B (Example 2-1) shows two overlays of all E1 (FIG. 4A) and E2 (FIG. 4B) fractions, collected during the intein purification cycles 1.1-1.5. FIG. 4C, 4D (Example 2-2) shows two overlays of all collected E1 (FIG. 4C) and E2 (FIG. 4D) fractions, collected during the intein purification cycles 2.1-2.5. The marked double-peak between 7 and 9 kDa was excluded from purity calculation due to unspecific background noise of the Denaturation LabChip® Sample Buffer. FIG. 4E shows the LabChip® Sample Buffer without proteins, for demonstrating the unspecific background noise, exemplary for the whole study.

FIG. 5 shows two different A280-Absorbance chromatograms of two intein purification cycles of Example 3. The figure demonstrates the A280 of the elution 1 (E1) and elution 2 (E2) fraction of two consecutive intein purification cycles using Capture Buffers A.1 and A.2. The intein-N ligand prototype column carrying R44-358132 was loaded two times with pre-purified intein-C tagged target solution (13 kDa), followed by eluting the target with two different Buffers (A.1, A.2).

FIG. 6 shows the eluted amount of target recovered from two intein purification cycles of Example 3. The intein-N ligand prototype column carrying R44-358132 was loaded twice with pre-purified intein-C tagged target solution (13 kDa) and the target was eluted using two different Capture Buffers for elution (A.1, A.2). The target amount of elution phase 1 (E1), phase 2 (E2) and the total amount of elution was calculated using A280-Absorbance and the appropriate extinction coefficient of the target protein.

FIG. 7: SDS Page analysis showing the protein composition of the elution step 1 (E1) and elution step 2 (E2) and CIP fractions of two intein purification cycles of Example 3 using two different elution buffers (A.1, A.2). E1: Elution fraction 1. E2: Elusion fraction 2. CIP: cleaning with 0.15 M H₃PO₄. A1: Capture Buffer pH 9. A2: Capture Buffer pH 9+0.3 M imidazole.

FIG. 8A-B shows an overlay of the fluorescence absorbance chromatograms of all elution fractions from two intein purification cycles of Example 3, analyzed by microfluidic electrophorese system. FIG. 8A shows the overlay of the elution (E1) fractions from two purification cycles. FIG. 8B shows the overlay of the elution (E2) fractions of the two purification cycles. The marked double-peak between 7 and 9 kDa was excluded from purity calculation due to unspecific background noise of the Denaturation LabChip® Sample Buffer (demonstrated in FIG. 4).

FIG. 9A-C shows different A280-Absorbance chromatogram overlays of in total five intein purification cycles of Example 4, using 0.2 ml/min flow rate (FIG. 9A: Cycle 1) and 1 ml/min flow rate in combination with 0.1 ml/min during the elution (FIG. 9B, 9C: Cycle 2-5). Two Cleavage Buffers B.1 (Reference, pH=7) and B.2 (Reference, pH=7+0.3 M imidazole) were used for the first three purification cycles 1, 2 and 3 (FIG. 9A, 9B). Two Capture Buffers A.1 (pH 9) and A.2 (+0.3 M imidazole) were used for purification cycle 4 and 5 (FIG. 9C). The intein-N ligand prototype column carrying R46-358132 was loaded with pre-purified intein-C tagged target solution. A 13 kDa target was used. The target was eluted using different elution buffers. The cleavage was triggered by addition of imidazole (buffers B.2 and A.2) as well as a pH shift (buffers B.1 and B.2).

FIG. 10A-B shows the amount of eluted target during five intein purification cycles of Example 4 using Cleavage Buffers B.1, B.2, A.1 and A.2 and 5 min (flow rate 0.2 ml/min) or minutes (flow rate 0.1 ml/min) residence time during the elution. FIG. 10A=Cycles 1-3. Cycle 1: Cleavage Buffer B.1 (pH 7), flow rate 0.2 ml/min Cycle 2: Cleavage Buffer B.1 (pH 7), flow rate 0.1 ml/min Cycle 3: Cleavage Buffer B.2 (pH 7, 0.3M imidazole), flow rate 0.1 ml/min. FIG. 10B=Cycles 4-5. Cycle 4: Capture Buffer A.1 (pH 9), flow rate 0.1 ml/min. Cycle 5: Capture Buffer A.2 (pH 9, 0.3M imidazole), flow rate 0.1 ml/min.

FIG. 11: SDS Page analysis showing the protein composition of the elution step 1 (E1) and elution step 2 (E2) and CIP fractions of two intein purification cycles of Example 4, using four different elution buffers (cycle 1 and 2): B.1, cycle 3: B.2, cycle 4: A.1, cycle 5: A.2). E1: Elution fraction 1. E2: Elusion fraction 2. CIP: cleaning with 0.15 M H₃PO₄. B 1: Cleavage Buffer pH 7. B2: Cleavage Buffer pH 7+0.3 M imidazole. A1: Capture Buffer pH 9. A2: Capture Buffer+0.3 M imidazole.

FIG. 12A-B shows an overlay of the fluorescence absorbance chromatograms of all elution fractions from five intein purification cycles (1-5) of Example 4, analyzed by microfluidic electrophorese system. FIGS. 12A and 12B shows the overlay of the elution (E1 and E2) fractions from purification cycles 1, 2 and 3. FIGS. 12C and 12D show the overlay of all elution (E1 and E2) fractions of the purification cycles 4 and 5. The marked double-peak between 7 and 9 kDa was excluded from purity calculation due to unspecific background noise of the Denaturation LabChip® Sample Buffer (demonstrated in FIG. 4).

FIG. 13: Overlay of the different A280-Absorbance chromatograms of intein purification cycle 1, 2, 3 and 4 within Example 5 with flow rate variation during the elution steps from 0.1 ml/min to 0.2 ml/min, 0.5 ml/min and 1 ml/min.

FIG. 14: Calculation of total bound, eluted and regenerated target during intein purification cycle 1, 2, 3 and 4. The elution phases were operated at a flow rate of 0.1/0.2/0.5 or 1 ml/min whereas the other process steps were set at 1 ml/min flow rate. The relative protein amount recovered from the elution and CIP phases in relation to the total bound protein is shown.

FIG. 15A-B: The two diagram cutouts show different A280-Absorbance chromatogram overlays of 4 intein purification cycles within Example 6, using different Cleavage Buffers B.1 or B.2 and two highly glycosylated target molecules: the 19 kDa hEPO within cycle 1 and 2 and the 26 kDa S1-RBD within cycle 3 and 4. The intein-N ligand prototype column, carrying R46-358132 (cycle 1 and 2, FIG. 15A), and prototype column carrying R49-358232 (cycle 3 and 4, FIG. 15B), was loaded with clarified cell lysate containing intein-C tagged target. In cycle 1 and 3, the target was eluted using Cleavage Buffers B.1 (100 mM Tris, 200 mM NaCl, pH 7), shown in solid black line. Alternatively, the target was eluted during cycle 2 and 4 using imidazole-enriched Cleavage Buffer B.2, demonstrated as a dashed line. The addition of 0.3 M (B.2) imidazole resulted in a higher amount of target elution that was recovered especially from elution phase 1.

FIG. 16 demonstrates the eluted target amount during cycle 1, 2, 3 and 4 of the intein purification using the intein-C tagged hEPO (cycle 1 and 2) and S1-RBD (cycle 3 and 4) and using Cleavage Buffers B.1 and B.2 within Example 6. The yield of the eluted hEPO was 12.22% higher, the eluted S1-RBD target was 1.95% higher if the Cleavage Buffer was enriched with 0.3 M imidazole.

FIG. 17: SDS Page analysis showing the protein composition of the clarified cell supernatant (CS), the wash (W), elution steps (E1, E2) and the CIP of four intein purification runs within Example 6 (Cycles 1-4), using two intein-N ligand prototype columns carrying R46-358132 (cycle 1 and 2) and R49-358132 (cycle 3 and 4). For both used proteins, the tagless released target at 19 or 26 kDa (hEPO within cycle 1 and 2 or S1-RBD within cycle 3 and 4) was observed, when elution was triggered with the different elution conditions (B.1 within cycle 1 and 3 or B.2 within cycle 2 and 4). Although the high glycosylation of the proteins led to a different running behavior on the SDS-Page, a higher protein size resulted than expected. The protein sizes were compared to the SigmaMarker™, 6.5-200 kDa (M1), or the Perfect Protein Marker, 10-225 kDa (M2).

### DETAILED DESCRIPTION

The present disclosure relates to methods of releasing a target molecule from intein complexes comprising an intein-C tagged target molecule and intein-N polypeptides, by contacting the intein complexes with nitrogen containing heteroaromatic derivatives, and/or by increasing residence time of intein complexes in a medium (e.g., solution) effective to remove the target molecule. Modulating the pH of a solution comprising the intein complex further facilitates target release.

### (1) Definitions

In order that the present disclosure may be more readily understood, certain terms are first defined. Additional definitions are set forth throughout the detailed description. Unless defined otherwise, all technical and scientific terms used herein have the same meaning as commonly understood by one of ordinary skill in the art to which this invention pertains.

The term “target molecule” as used herein refers to a biological molecule (e.g., protein), material or macromolecular assembly, which is to be, e.g., purified or removed from a mixture (e.g., a crude protein mixture). Exemplary target molecules include, for example, recombinant peptides and proteins, including antibodies (e.g., monoclonal antibodies), vaccines, viruses, and other macromolecular assemblies, such as virus-like particles and nanoparticles that may incorporate both biomolecular and synthetic components. By way of example, target molecules can include proteins and biomolecular assemblies (e.g., produced by recombinant DNA technology), such as, e.g., hormones (e.g. insulin, human growth hormone, erythropoietin, interferons, granulocyte colony stimulating factor, tissue plasminogen activator), monoclonal antibodies (mAbs) and mAb-derivatives (e.g., bi-specific mAbs, Fabs, scFvs, shark and camelid antibodies), scaffold-derived therapeutics (e.g., DARPins, Affibodies, anticalins), therapeutic enzymes (e.g., alpha galactosidase A, alpha-L-iduronidase, N-acetylgalactosamine-4-sulfatase, glucocerebrosidase), toxins (e.g. botulinum, CRM 197, ricin), recombinant vaccines (e.g., anthrax, diphtheria, tetanus, pneumonia, hepatitis B virus, human papilloma virus), virus-like particles (e.g., hepatitis B, human papilloma, influenza, parvovirus, Norwalk viruses), as well as industrial enzymes (e.g., papain, bromelain, trypsin, proteinase K, BENZONASE®. enzyme, DENERASE™ enzyme, urease, pepsin, and the like) diagnostic reagents (e.g., glucose and lactate dehydrogenase, DNA polymerases, alkaline phosphatase, horseradish peroxidase, restriction enzymes, hybridoma-derived antibodies and the like), and viral vectors (e.g., Lenti Virus vector, Adeno Associated Virus (AAV) vector, herpex simplex-1 viral vector (HSV-1), and the like).

The term “fusion protein” as used herein refers to a naturally occurring, synthetic, semi-synthetic or recombinant single protein molecule that comprises all or a portion of two or more heterologous polypeptides joined by peptide bonds.

The term “peptide”, “peptidic”, as used herein, refers to peptides and proteins longer than two amino acids in length that may also incorporate non-amino acid molecules.

The term “polypeptide” refers to a polymer of amino acids, and not to a specific length; thus, peptides, oligopeptides and proteins are included within the definition of a polypeptide.

The term “intein”, as used herein, refers to a protein, either isolated from nature or created through recombinant DNA technology, with autocatalytic activity. Inteins contain internal sequences or segments that may be spliced out of the larger molecule after it is translated, leaving the remaining segments (the “exteins”) to rejoin and form a new protein

The term “split intein”, as used herein, refers to a protein, either isolated from nature or created through recombinant DNA technology, that has the following properties: (1) the protein occurs in two halves that interact with high affinity and selectivity; (2) the two halves must contain all intein sequences required for catalytic activity and may also contain appended non-intein-N peptidic sequences; (3) the protein has enzymatic activity only when the two halves are tightly associated; and (4) the enzymatic activity is site selective peptidic cleavage or ligation that serves to separate intein sequences from non-intein-N peptidic sequences or ligate the non-intein-N peptidic sequences into contiguous linear or circular proteins.

The term “complementary inteins” is used herein to refer to the intein-N and intein-C portions of a split intein pair.

The term “intein-N”, as used herein, refers to an intein polypeptide having homology to the N-terminal portion of a single intein polypeptide, and which associates with a complementary intein-C to form an active intein enzyme.

The term “intein-C”, as used herein, refers to an intein polypeptide having homology to the C-terminal portion of a single intein polypeptide, and which associates with a complementary intein-N to form an active intein enzyme.

The term “extein”, as used herein, refers to N- and C-terminal peptidic sequences that are fused to N- and intein-Cs in nature and are manipulated (e.g., cleaved or ligated) through the enzymatic action of the split intein.

The term “chromatography,” as used herein, refers to a dynamic separation technique which separates a target molecule of interest from other molecules in the mixture and allows it to be isolated. Typically, in a chromatography method, a mobile phase (liquid or gas) transports a sample containing the target molecule of interest across or through a stationary phase (normally solid) medium. Differences in partition or affinity to the stationary phase separate the different molecules while mobile phase carries the different molecules out at different time.

The term “affinity chromatography,” as used herein, refers to a mode of chromatography where a target molecule to be separated is isolated by its interaction with a molecule (e.g., an affinity chromatography ligand according to this invention comprising an intein-N and intein-N solubilization factor) which specifically interacts with the target molecule. In one embodiment, affinity chromatography involves the addition of a sample containing a target molecule (e.g., a protein) to a solid support which carries on it an intein-N-based ligand, as described herein.

### (2) Description of the Process of the Invention

The present invention relates to a method for releasing a target molecule from an intein complex comprising (i) a fusion protein comprising an intein-C polypeptide joined to the target molecule by a peptide bond (intein-C tagged target molecule); and (ii) an intein-N polypeptide. The method comprising the steps of:
(a) contacting the intein-C tagged target molecule with the intein-N polypeptide, for a first time period sufficient to form the intein complex; and
(b) releasing the target molecule from the intein complex by:
   (i) contacting the intein complex with a medium effective to remove the target molecule, for a second time period which is longer than the first time period; and/or
   (ii) contacting the intein complex with a heteroaromatic compound comprising at least one ring nitrogen atom.

The methods of the present disclosure are applicable to a variety of intein-mediated processes. In some embodiments, the intein-mediated process is protein purification. In other embodiments the intein-mediated process is protein ligation. In other embodiments, the intein-mediated process is in vivo protein tagging. In other embodiments, the intein-mediated process is protein labelling. In other embodiments, the intein-mediated process is protein cyclization. In other embodiments, the intein-mediated process is protein polymerization. In other embodiments, the intein-mediated process is intein-induced reporter pathway analysis. In other embodiments the intein-mediated process is a process for preparation of fusion proteins.

The methods of the present disclosure are applicable to a variety of intein-tagged molecules, e.g., intein-C tagged molecule. In one embodiment, the intein-C tagged molecule is a protein. In one embodiment, the intein-C tagged molecule is a polypeptide. In another embodiment, the intein-C tagged molecule is a glycosylated protein. In another embodiment, the intein-C tagged molecule is a highly glycosylated protein.

As used herein, the term “glycosylation” means a post-translational modification whereby sugar moieties (e.g., monosaccharides, disaccharides or oligosaccharides) are attached to proteins to form glycosidic or glycopeptide bonds. Glycopeptide bonds can be categorized into different groups based on the nature of the sugar-peptide bond and the saccharide attached. Examples of glycosylation include (a) N-linked glycosylation comprising binding of a sugar to the amino group of asparagine; (b) O-linked glycosylation comprising binding of a sugar to hydroxyl group of serine or threonine; (c) C-linked glycosylation comprising binding of a sugar to the indole ring of tryptophan; and (d) glypiation comprising binding of a protein and phospholipid via a sugar moiety. The glycosylated protein used in the methods of the present disclosure may comprise any one or more of these glycosylation pattern.

As used herein, the term “highly glycosylated protein” refers to a protein comprising at least about 10% glycosylated amino acids. In some embodiments, the protein comprising at least about 20% glycosylated amino acids, at least about 25% glycosylated amino acids, at least about 30% glycosylated amino acids, at least about 35% glycosylated amino acids, at least about 40% glycosylated amino acids, at least about 45% glycosylated amino acids, at least about 50% glycosylated amino acids, at least about 55% glycosylated amino acids, at least about 60% glycosylated amino acids, at least about 65% glycosylated amino acids, at least about 70% glycosylated amino acids, at least about 75% glycosylated amino acids, at least about 80% glycosylated amino acids, at least about 85% glycosylated amino acids, at least about 90% glycosylated amino acids, or at least about 95% glycosylated amino acids.

In some embodiments, the intein-C tagged molecule is an intein-C tagged protein. In some embodiments, the intein-C tagged protein has a molecular weight (MW) of about 1 kDa to about 100 kDa including the intein-C tag. In other embodiments, the intein-C tagged protein has a molecular weight (MW) of about 10 kDa to about 100 kDa including the intein-C tag. In other embodiments, the intein-C tagged protein has a molecular weight (MW) of about 10 kDa to about 75 kDa including the intein-C tag. In other embodiments, the intein-C tagged protein has a molecular weight (MW) of about 10 kDa to about 50 kDa including the intein-C tag. In other embodiments, the intein-C tagged protein has a molecular weight (MW) of about 10 kDa to about 40 kDa including the intein-C tag. In other embodiments, the intein-C tagged protein has a molecular weight (MW) of about 20 kDa to about 50 kDa including the intein-C tag. In other embodiments, the intein-C tagged protein has a molecular weight (MW) of about 20 kDa to about 40 kDa including the intein-C tag. In other embodiments, the intein-C tagged protein has a molecular weight (MW) of about 10 kDa to about 15 kDa including the intein-C tag. In other embodiments, the intein-C tagged protein has a molecular weight (MW) of about 10 kDa to about 20 kDa including the intein-C tag. In other embodiments, the intein-C tagged protein has a molecular weight (MW) of about 10 kDa to about 25 kDa including the intein-C tag. In other embodiments, the intein-C tagged protein has a molecular weight (MW) of about 10 kDa to about 30 kDa including the intein-C tag. In other embodiments, the intein-C tagged protein has a molecular weight (MW) of about 10 kDa to about 35 kDa including the intein-C tag. In other embodiments, the intein-C tagged protein has a molecular weight (MW) of about 20 kDa to about 40 kDa including the intein-C tag. In other embodiments, the intein-C tagged protein has a molecular weight (MW) of about 25 kDa to about 40 kDa including the intein-C tag. In other embodiments, the intein-C tagged protein has a molecular weight (MW) of about 30 kDa to about 40 kDa including the intein-C tag. In other embodiments, the intein-C tagged protein has a molecular weight (MW) of about 35 kDa to about 40 kDa including the intein-C tag. In other embodiments, the intein-C tagged protein has a molecular weight (MW) of about 10 kDa, about 11 kDa, about 12 kDa, about 13 kDa, about 14 kDa, about 15 kDa, about 16 kDa, about 17 kDa, about 18, kDa, about 19 kDa, about 20 kDa, about 21 kDa, about 22 kDa, about 23 kDa, about 24 kDa, about 25 kDa, about 26 kDa, about 27 kDa, about 28, kDa, about 29 kDa, about 30 kDa, about 31 kDa, about 32 kDa, about 33 kDa, about 34 kDa, about 35 kDa, about 36 kDa, about 37 kDa, about 38, kDa, about 39 kDa, about 40 kDa, about 41 kDa, about 42 kDa, about 43 kDa, about 44 kDa, about 45 kDa, about 46 kDa, about 47 kDa, about 48, kDa, about 49 kDa, about 50 kDa, including the intein-C tag.

In some embodiments, the intein-C tagged protein has a molecular weight (MW) of about 1 kDa to about 100 kDa excluding the intein-C tag. In other embodiments, the intein-C tagged protein has a molecular weight (MW) of about 10 kDa to about 100 kDa excluding the intein-C tag. In other embodiments, the intein-C tagged protein has a molecular weight (MW) of about 10 kDa to about 75 kDa excluding the intein-C tag. In other embodiments, the intein-C tagged protein has a molecular weight (MW) of about 10 kDa to about 50 kDa excluding the intein-C tag. In other embodiments, the intein-C tagged protein has a molecular weight (MW) of about 10 kDa to about 40 kDa excluding the intein-C tag. In other embodiments, the intein-C tagged protein has a molecular weight (MW) of about 20 kDa to about 50 kDa excluding the intein-C tag. In other embodiments, the intein-C tagged protein has a molecular weight (MW) of about 20 kDa to about 40 kDa excluding the intein-C tag. In other embodiments, the intein-C tagged protein has a molecular weight (MW) of about 10 kDa to about 15 kDa excluding the intein-C tag. In other embodiments, the intein-C tagged protein has a molecular weight (MW) of about 10 kDa to about 20 kDa excluding the intein-C tag. In other embodiments, the intein-C tagged protein has a molecular weight (MW) of about 10 kDa to about 25 kDa excluding the intein-C tag. In other embodiments, the intein-C tagged protein has a molecular weight (MW) of about 10 kDa to about 30 kDa excluding the intein-C tag. In other embodiments, the intein-C tagged protein has a molecular weight (MW) of about 10 kDa to about 35 kDa excluding the intein-C tag. In other embodiments, the intein-C tagged protein has a molecular weight (MW) of about 20 kDa to about 40 kDa excluding the intein-C tag. In other embodiments, the intein-C tagged protein has a molecular weight (MW) of about 25 kDa to about 40 kDa excluding the intein-C tag. In other embodiments, the intein-C tagged protein has a molecular weight (MW) of about 30 kDa to about 40 kDa excluding the intein-C tag. In other embodiments, the intein-C tagged protein has a molecular weight (MW) of about 35 kDa to about 40 kDa excluding the intein-C tag. In other embodiments, the intein-C tagged protein has a molecular weight (MW) of about 10 kDa, about 11 kDa, about 12 kDa, about 13 kDa, about 14 kDa, about 15 kDa, about 16 kDa, about 17 kDa, about 18, kDa, about 19 kDa, about 20 kDa, about 21 kDa, about 22 kDa, about 23 kDa, about 24 kDa, about 25 kDa, about 26 kDa, about 27 kDa, about 28, kDa, about 29 kDa, about 30 kDa, about 31 kDa, about 32 kDa, about 33 kDa, about 34 kDa, about 35 kDa, about 36 kDa, about 37 kDa, about 38, kDa, about 39 kDa, about 40 kDa, about 41 kDa, about 42 kDa, about 43 kDa, about 44 kDa, about 45 kDa, about 46 kDa, about 47 kDa, about 48, kDa, about 49 kDa, about 50 kDa, excluding the intein-C tag.

### N-heteroaromatic Additives

In one aspect, it has surprisingly been found that target release from intein complexes comprising a covalently-bound target molecule (e.g., proteins) can be controlled by addition of nitrogen-containing heteroaromatic compounds such as azoles (e.g., imidazole, pyrazole, oxazole) and azole-containing compounds (e.g., histidine).

As contemplated herein, it has now been shown that addition of certain catalytic additives, namely nitrogen-containing heteroaromatic compounds facilitates and catalyzes target release from intein complexes. With such catalytic additives, it is possible to control and catalyze the enzymatic intein reaction leading to faster splicing/cleavage rates and a higher yield of target release. By way of exemplification and not for limitation, this behaviour was found to be consistent throughout a wide variety of additives and intein-N fragments immobilized on chromatographic support.

Thus, in one embodiment, the present invention relates to a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of contacting the intein complex with a heteroaromatic compound comprising at least one ring nitrogen atom.

In another embodiment, the present invention relates to a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of: (a) contacting the intein-C tagged target molecule with the intein-N polypeptide, for a first time period sufficient to form the intein complex; and (b) releasing the target molecule from the intein complex by contacting the intein complex with a heteroaromatic compound comprising at least one ring nitrogen atom. Optionally, the release step (b) is performed for a second time period which is longer than the first time period.

In some embodiments, the intein complex is formed during an intein-mediated process selected from the group consisting of protein purification, protein ligation, in vivo protein tagging, protein labelling, protein cyclization, protein polymerization, intein-induced reporter pathway analysis, and preparation of fusion proteins.

In other embodiments, the step of releasing the target molecule further involves reducing the pH of the medium. For example, when the process involves protein purification, the pH of the loading solution is reduced to facilitate elution of the target molecule.

In some embodiments, the heteroaromatic compound is an azole or azole-containing compound.

In other embodiments, the heteroaromatic compound is selected from the group consisting of an unsubstituted or substituted imidazole, pyrazole, 1,2,3-triazole, 1,2,4-triazole, tetrazole, pentazole, oxazole, isoxazole, 1,2,3-oxadiazole, 1,2,4-oxadiazole, furzan (1,2,5-oxadiazole), 1,3,4-oxadiazole, thiazole, isothiazole, thiadiazole (1,2,3-tiadiazole), 1,2,4-thiadaizole 1,2,5-thiadiazole, 1,3,4-thiadiazole, histidine, pyridine, pyrazine, pyrrole, pyrimidine, pyridazine, and any combination thereof.

In some embodiments, the method of the present disclosure is used for purifying a target molecule. In accordance with this embodiment, the method comprises the steps of:
(a) contacting the intein-C tagged target molecule with the intein-N polypeptide on a chromatography resin at a first flow rate so as to form the intein complex; and
(b) releasing the target molecule from the intein complex in the presence of a heteroaromatic compound comprising at least one ring nitrogen atom. Optionally, step (b) is performed at a second flow rate that is slower than the first flow rate.

In some embodiments, the method comprises the steps of:
(a) providing a sample containing the intein-C tagged target molecule;
(b) loading the sample on a column comprising a chromatography resin, the chromatography resin comprising a covalently-linked N-terminal intein polypeptide, under conditions in which the intein-C polypeptide in the fusion protein binds to the intein-N polypeptide in the resin to form an intein complex, wherein the sample is loaded at a first flow rate/column residence time;
(c) optionally washing the resin containing the intein complex to remove unbound contaminants;
(d) cleaving the target molecule from the intein complex by contacting the intein complex with a heteroaromatic compound comprising at least one ring nitrogen atom; optionally wherein step (d) is performed at a second flow rate that is slower than the first flow rate, or a second column residence time that is longer than the first column residence time;
(e) regenerating the chromatography resin;
(f) optionally, performing at least one additional purification cycle by repeating steps (a) to (e) at least once; and
(g) optionally, isolating the target molecule.

In some embodiments, step (d) is performed at a lower pH than step (b) and optional step (c). Preferably, step (b) comprises loading the intein-C tagged target molecule in a saline buffer having a pH of about 8 to about 10, more preferably a pH of about 9; and step (d) comprises contacting the intein complex with a saline buffer having a pH of about 6 to about 8, more preferably a pH of about 7.

In some embodiments, step (d) is performed at about the same pH as step (b) and optional step (c). Preferably, steps (b), optional step (c) and step (d) are each performed in a saline buffer having a pH of about 8 to about 10, and more preferably, steps (b), optional step (c) and step (d) are each performed at a pH of about 9.

In some embodiments, each of steps (b), (c) (if performed), (d) and (e) is independently performed under static incubation or constant flow representing residence times of 0.1-120 min per Column Volume (CV).

In some embodiments, step (b) comprises contacting the chromatography resin with a cell culture supernatant comprising the intein-C tagged target molecule.

In some embodiments, step (c) is performed, and comprises washing the chromatography resin with a washing buffer prior to releasing the target molecule from the intein-C polypeptide; preferably wherein the washing buffer comprises a detergent, a salt, a chaotropic agent, preferably urea or arginine, or a combination thereof.

In some embodiments, the intein-N polypeptide is attached to the chromatography resin through a functional group selected from the group consisting of hydroxyl, thiol, epoxide, amino, carbonyl epoxide and carboxylic acid.

In some embodiments, the second flow rate, if implemented, is at least about 2 times slower than the first flow rate, preferably between about 2 to about 20 times slower, more preferably between about 2 to about 10 times slower, and most preferably between about 5 to about 10 times slower. In other embodiments, the second column residence time, if implemented, is at least about 2 times longer than the first column residence time, preferably between about 2 to about 20 times longer, more preferably between about 2 to about 10 times longer, and most preferably between about 5 to about 10 times longer.

In some embodiments, the increase in contact time, the presence of the heteroaromatic compound and/or the reduction in pH during the target molecule release step increases the yield of the target molecule by at least about 1%, or by at least about 2%, or by at least about 3%, or by at least about 5%, or by at least about 10%, or by at least about 15%, or by at least about 20%, or by at least about 25%. In other embodiments, the increase in contact time, the presence of the heteroaromatic compound and/or the reduction in pH during the target molecule release step increases the yield of the target molecule collected during elution step 1 (E1) by at least about 1%, or by at least about 2%, or by at least about 3%, or by at least about 5%, or by at least about 10%, or by at least about 15%, or by at least about 20%, or by at least about 25%, or by at least about 30%, or by at least about 35%, or by at least about 40%.

As demonstrated herein, in some embodiments, the presence of the heteroaromatic compound increases the overall yield of the target molecule by at least about 1%, or by at least about 2%, or by at least about 3%, or by at least about 5%, or by at least about 10%, or by at least about 15%, or by at least about 20% or by at least about 25%. In other embodiments, the presence of the heteroaromatic compound increases the yield of the target molecule collected during elution step 1 (E1) by at least about 1%, or by at least about 2%, or by at least about 3%, or by at least about 5%, or by at least about 10%, or by at least about 15%, or by at least about 20%, or by at least about 25%, or by at least about 30%, or by at least about 35%, or by at least about 40%.

As demonstrated herein, in some embodiments, a pH shift (i.e., reduction in pH) increases the overall yield of the target molecule by at least about 1%, or by at least about 2%, or by at least about 3%, or by at least about 5%, or by at least about 10%, or by at least about 15%, or by at least about 20%, or by at least about 25%. In other embodiments, a pH shift (i.e., reduction in pH) increases the yield of the target molecule collected during elution step 1 (E1) by at least about 1%, or by at least about 2%, or by at least about 3%, or by at least about 5%, or by at least about 10%, or by at least about 15%, or by at least about 20%, or by at least about 25%, or by at least about 30%, or by at least about 35%, or by at least about 40%.

As demonstrated herein, in some embodiments, increase in contact time increases the overall yield of the target molecule by at least about 1%, or by at least about 2%, or by at least about 3%, or by at least about 5%, or by at least about 10%, or by at least about 15%, or by at least about 20%, or by at least about 25%. In other embodiments, increase in contact time increases the yield of the target molecule collected during elution step 1 (E1) by at least about 1%, or by at least about 2%, or by at least about 3%, or by at least about 5%, or by at least about 10%, or by at least about 15%, or by at least about 20%, or by at least about 25%, or by at least about 30%, or by at least about 35%, or by at least about 40%.

In some embodiments, the target molecule is a protein. Preferably, the sample is a crude protein preparation.

As used herein, the term “heteroaromatic compound comprising at least one ring nitrogen atom” or “heteroaryl comprising at least one nitrogen atom” or “N-containing heteroaromatic compound”, used herein interchangeably, means a compound comprising a heteroaromatic system containing at least one ring nitrogen (N) atom, and optionally additional nitrogen, sulfur and oxygen atoms. The heteroaryl ring preferably contains 5 or more ring atoms. The heteroaryl group can be monocyclic, bicyclic, tricyclic and the like. Also included in this definition are the benzoheteroaromatic rings. The present disclosure also contemplates the N-oxides of the nitrogen containing heteroaryls. The present disclosure also contemplates salts of the nitrogen containing heteroaryls.

Non-limiting examples of N-containing heteroaromatic compounds include unsubstituted or substituted imidazole, pyrazole, 1,2,3-triazole, 1,2,4-triazole, tetrazole, pentazole, oxazole, isoxazole, 1,2,3-oxadiazole, 1,2,4-oxadiazole, furzan (1,2,5-oxadiazole), 1,3,4-oxadiazole, thiazole, isothiazole, thiadiazole (1,2,3-tiadiazole), 1,2,4-thiadaizole 1,2,5-thiadiazole, 1,3,4-thiadiazole, histidine, pyridine, pyrazine, pyrrole, pyrimidine, pyridazine, and any combination thereof. The heteroaryl group can be unsubstituted or substituted through available atoms with one or more groups including but not limited to halogen, hydroxy, alkoxy carbonyl, amido, alkylamido, dialkylamido, nitro, amino, alkylamino, dialkylamino, carboxyl, thio and thioalkyl.

In one embodiment, the nitrogen-containing heteroaromatic compound is an azole or incorporates and azole moiety. An “azole” as used herein means a five-membered heterocyclic compound containing a nitrogen atom and at least one other non-carbon atom (i.e. nitrogen, sulfur, or oxygen) as part of the ring. In one embodiment, the compound is an azole. In another embodiment, the compound is an azole derivative, i.e., a compound which incorporates an azolyl moiety. In one embodiment, the azole is imidazole. In another embodiment, the azole is pyrazole. In another embodiment, the azole is histidine, i.e., an amino acid comprising an imidazole side chain.

Preferably, the additive/heteroaromatic compound is provided at a concentration effective to facilitate or increase the rate/yield of target molecule release from the intein complex. When applied to a chromatographic separation technique, the additive/heteroaromatic compound is preferably dissolved in a solution and passed through a chromatographic system at a concentration and rate sufficient to elute the target molecule from an intein complex from the chromatographic support.

In one embodiment, the heteroaromatic compound heteroaromatic compound is added at a concentration of between about 1 mM to about 1 M. In another embodiment, the heteroaromatic compound is added at a concentration of between about 5 mM to about 1 M. In another embodiment, the heteroaromatic compound is added at a concentration of between about 10 mM to about 1 M. In another embodiment, the heteroaromatic compound is added at a concentration of between about 10 mM to about 1 M. In another embodiment, the heteroaromatic compound is added at a concentration of between about 100 mM to about 1 M. In another embodiment, the heteroaromatic compound is added at a concentration of between about 100 mM to about 750 mM. In another embodiment, the heteroaromatic compound is added at a concentration of between about 100 mM to about 600 mM. In another embodiment, the heteroaromatic compound is added at a concentration of about 100 mM. In another embodiment, the heteroaromatic compound is added at a concentration of about 200 mM. In another embodiment, the heteroaromatic compound is added at a concentration of about 300 mM. In another embodiment, the heteroaromatic compound is added at a concentration of about 400 mM. In another embodiment, the heteroaromatic compound is added at a concentration of about 500 In another embodiment, the heteroaromatic compound is added at a concentration of about 600 mM. In another embodiment, the heteroaromatic compound is added at a concentration of about 700 mM. In another embodiment, the heteroaromatic compound is added at a concentration of about 750 mM. In another embodiment, the heteroaromatic compound is added at a concentration of about 800 mM. In another embodiment, the heteroaromatic compound is added at a concentration of about 900 mM. It is apparent to a person of skill in the art that the specification concentration of the additive may vary depending on the reaction, the nature of the target molecule and the nature of the column use. The appropriate concentration may be determined by a person of skill in the art.

### Modulation of Contact Time

In another aspect, it has now been discovered that extending the residence time of an intein complex in a medium (e.g., solution, suspension) effective to release the target molecule, as compared with the residence time utilized to form the complex, facilitates target cleavage. Reduction of the pH of the medium during target removal further enhances release of the target molecule. With these manipulations, the enzymatic release reactions can be controlled leading to faster splicing/cleavage rates and higher yields of target release.

Thus, in one embodiment, the present invention relates to a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of: (a) contacting the intein-C tagged target molecule with the intein-N polypeptide, for a first time period sufficient to form the intein complex; and (b) releasing the target molecule from the intein complex by contacting the intein complex with a medium effective to remove the target molecule, for a second time period which is longer than the first time period. Optionally, step (b) is performed in the presence of a heteroaromatic compound comprising at least one ring nitrogen atom.

In some embodiments, the intein complex is formed during an intein-mediated process selected from the group consisting of protein purification, protein ligation, in vivo protein tagging, protein labelling, protein cyclization, protein polymerization, intein-induced reporter pathway analysis, and preparation of fusion proteins.

In other embodiments, the step of releasing the target molecule further involves reducing the pH of the medium. For example, when the process involves protein purification, the pH of the loading solution is reduced to facilitate elution of the target molecule.

In some embodiments, the optional heteroaromatic compound is an azole or azole-containing compound. In other embodiments, the optional heteroaromatic compound is selected from the group consisting of an unsubstituted or substituted imidazole, pyrazole, 1,2,3-triazole, 1,2,4-triazole, tetrazole, pentazole, oxazole, isoxazole, 1,2,3-oxadiazole, 1,2,4-oxadiazole, furzan (1,2,5-oxadiazole), 1,3,4-oxadiazole, thiazole, isothiazole, thiadiazole (1,2,3-tiadiazole), 1,2,4-thiadaizole 1,2,5-thiadiazole, 1,3,4-thiadiazole, histidine, pyridine, pyrazine, pyrrole, pyrimidine, pyridazine, and any combination thereof.

In some embodiments, the method of the present disclosure is used for purifying a target molecule. In accordance with this embodiment, the method comprises the steps of:
(a) contacting the intein-C tagged target molecule with the intein-N polypeptide on a chromatography resin at a first flow rate so as to form the intein complex; and
(b) releasing the target molecule from the intein complex by contacting the intein complex with a medium effective to remove the target molecule, for a second time period which is longer than the first time period. Optionally, step (b) is performed in the presence of a heteroaromatic compound comprising at least one ring nitrogen atom.

In some embodiments, the method comprises the steps of:
(a) providing a sample containing the intein-C tagged target molecule;
(b) loading the sample on a column comprising a chromatography resin, the chromatography resin comprising a covalently-linked N-terminal intein polypeptide, under conditions in which the intein-C polypeptide in the fusion protein binds to the intein-N polypeptide in the resin to form an intein complex, wherein the sample is loaded at a first flow rate/column residence time;
(c) optionally washing the resin containing the intein complex to remove unbound contaminants;
(d) cleaving the target molecule from the intein complex by contacting the intein complex with a medium effective to cleave the target molecule at a second flow rate that is slower than the first flow rate, or a second column residence time that is longer than the first column residence time, wherein step (d) is optionally performed in the presence of a heteroaromatic compound comprising at least one ring nitrogen atom.
(e) regenerating the chromatography resin;
(f) optionally, performing at least one additional purification cycle by repeating steps (a) to (e) at least once; and
(g) optionally, isolating the target molecule.

In some embodiments, step (d) is performed at a lower pH than step (b) and optional step (c). Preferably, step (b) comprises loading the intein-C tagged target molecule in a saline buffer having a pH of about 8 to about 10, more preferably a pH of about 9; and step (d) comprises contacting the intein complex with a saline buffer having a pH of about 6 to about 8, more preferably a pH of about 7.

In some embodiments, step (d) is performed at about the same pH as step (b) and optional step (c). Preferably, steps (b), optional step (c) and step (d) are each performed in a saline buffer having a pH of about 8 to about 10, and more preferably, steps (b), optional step (c) and step (d) are each performed at a pH of about 9.

In some embodiments, each of steps (b), (c) (if performed), (d) and (e) is independently performed under static incubation or constant flow representing residence times of 0.1-120 min per Column Volume (CV).

In some embodiments, step (b) comprises contacting the chromatography resin with a cell culture supernatant comprising the intein-C tagged target molecule.

In some embodiments, step (c) is performed, and comprises washing the chromatography resin with a washing buffer prior to releasing the target molecule from the intein-C polypeptide; preferably wherein the washing buffer comprises a detergent, a salt, a chaotropic agent, preferably urea or arginine, or a combination thereof.

In some embodiments, the intein-N polypeptide is attached to the chromatography resin through a functional group selected from the group consisting of hydroxyl, thiol, epoxide, amino, carbonyl epoxide and carboxylic acid.

In some embodiments, the second flow rate is at least about 2 times slower than the first flow rate, preferably between about 2 to about 20 times slower, more preferably between about 2 to about 10 times slower, and most preferably between about 5 to about 10 times slower. In other embodiments, the second column residence time is at least about 2 times longer than the first column residence time, preferably between about 2 to about 20 times longer, more preferably between about 2 to about 10 times longer, and most preferably between about 5 to about 10 times longer.

In some embodiments, the increase in contact time, the presence of the heteroaromatic compound and/or the reduction in pH during the target molecule release step increases the yield of the target molecule by at least about 5%, preferably at least about 10%, and more preferably by at least about 20%, and even more preferably by at least about 50%.

When the methods of the present disclosure are applied to protein purification on columns, the contact time may be modified by changing the flow rate of the column, and/or manipulating the residence time of the eluting solution.

### Protein Purification

In one particular embodiment, the intein complex is formed as part of a protein purification process. In accordance with this embodiment, the process involves affinity chromatography for purifying a target biological molecule, utilizing intein-N ligands covalently bound on a chromatography resin, which is preferably attached to a solid support. Intein-C tagged proteins are passed through the column under conditions sufficient to form a stable complex between the intein-N fragment and the intein-C fragment. After an optional washing step to remove process contaminants, tagless release of the target is triggered by a change in the flow rate/column residence time and/or addition of a heteroaromatic compound comprising at least one ring nitrogen atom ((e.g., azole or azole containing compound) and/or a change in the pH. Finally, the protein is regenerated by disrupting the intein-N and intein-C complex and regenerating the intein N-resin. Optionally the process is repeated in multiple cycles as needed.

In accordance with this embodiment, an intein complex comprising a covalently tagged target molecule is formed by loading an intein-C tagged target molecule onto a column comprising a chromatography resin having an intein-N polypeptide immobilized thereto. The reaction is operated at a column flow rate sufficient to allow intein-N polypeptide to react with the intein-C tagged molecule to form an intein complex. The target molecule is then cleaved and released from the intein complex and eluted out of the column It has now been unexpectedly found that modulating (i.e., slowing) the flow rate of the eluting solution facilitates cleavage and release of the target molecule.

Thus, in one embodiment, the present disclosure further relates to a method for purifying a target molecule, the method comprising the step of: (a) contacting the intein-C tagged target molecule with the intein-N polypeptide on a chromatography resin at a first flow rate so as to form the intein complex; and (b) releasing the target molecule from the intein complex at a second flow rate which is slower than the first flow rate.

In one embodiment, the second flow rate is at least about 2 times slower than the first flow rate. In another embodiment, the second flow rate is between about 2 to about 20 times slower. In another embodiment, the second flow rate is between about 2 to about 10 times slower than the first flow rate. In another embodiment, the second flow rate is between about 5 to about 10 times slower than the first flow rate. In another embodiment, the second flow rate is about 2-5 times slower than the first flow rate. In another embodiment, the second flow rate is about 5-8 times slower than the first flow rate. In another embodiment, the second flow rate is about 8-10 times slower than the first flow rate. In another embodiment, the second flow rate is about 10-12 times slower than the first flow rate. In another embodiment, the second flow rate is about 12-14 times slower than the first flow rate. In another embodiment, the second flow rate is about 14-16 times slower than the first flow rate. In another embodiment, the second flow rate is about 16-18 times slower than the first flow rate. In another embodiment, the second flow rate is about 18-20 times slower than the first flow rate. It is apparent to a skill in the art that the ratio between the first and second flow rates can be determined by a person of skill in the art depending on the nature of the column, the target molecule used and other reaction conditions.

By way of example, the first flow rate is at least about 1 ml/min, and the second flow rate is between about 0.1 ml/min to about 0.9 ml/min. In another embodiment, the first flow rate is at least about 1 ml/min, and the second flow rate is between about 0.1 ml/min to about ml/min In another embodiment, the first flow rate is at least about 1 ml/min, and the second flow rate is between about 0.1 ml/min and 0.2 ml/min.

Furthermore, it is apparent to a person of skill in the art that the flow rate of the column is inversely proportional to the column residence time, i.e., the time it takes the eluting solution to pass through a column volume (CV). The faster the flow rate, the shorter the column residence time and vice versa. Thus, in accordance with alternative aspects of the present disclosure, increasing the column residence time of the eluting solution facilitates cleavage and release of the target molecule.

Thus, in one embodiment, the present disclosure further relates to a method for purifying a target molecule, the method comprising the step of (a) contacting the intein-C tagged target molecule with the intein-N polypeptide on a chromatography resin at a first column residence time so as to form the intein complex; and (b) releasing the target molecule from the intein complex at a column residence time which is longer than the first column residence time.

In one embodiment, the second column residence time is at least about 2 times longer than the first column residence time. In another embodiment, the second column residence time is between about 2 to about 20 times longer than the first column residence time. In another embodiment, the second column residence time is between about 2 to about 10 times longer than the first column residence time. In another embodiment, the second column residence time is between about 5 to about 10 times longer than the first column residence time. In one embodiment, the second column residence time is at least about 4 times longer than the first column residence time. In one embodiment, the second column residence time is at least about 6 times longer than the first column residence time. In one embodiment, the second column residence time is at least about 8 times longer than the first column residence time. In one embodiment, the second column residence time is at least about 10 times longer than the first column residence time. In one embodiment, the second column residence time is at least about 12 times longer than the first column residence time. In one embodiment, the second column residence time is at least about 14 times longer than the first column residence time. In one embodiment, the second column residence time is at least about 16 times longer than the first column residence time. In one embodiment, the second column residence time is at least about 18 times longer than the first column residence time. In one embodiment, the second column residence time is at least about 20 times longer than the first column residence time.

By way of exemplification, the first column residence time may be about 0.1 to about minutes per Column Volume (CV) or shorter, and the second residence time may be between about 2 minutes to about 100 minutes per Column Volume (CV) or longer. In some embodiments, the first column residence time may be about 0.1 to about 5 minutes per Column Volume (CV) or shorter, and the second residence time may be between about 2 minutes to about 100 minutes per Column Volume (CV) or longer. In some embodiments, the first column residence time may be about 0.1 to about 1 minutes per Column Volume (CV) or shorter, and the second residence time may be between about 2 minutes to about 100 minutes per Column Volume (CV) or longer. In some embodiments, the first column residence time may be about 0.1 to about 1 minute per Column Volume (CV) or shorter, and the second residence time may be between about 2 minutes to about 10 minutes per Column Volume (CV) or longer. In some embodiments, the first column residence time may be about 0.1 to about 1 minute per Column Volume (CV) or shorter, and the second residence time may be between about 2 minutes to about 10 minutes per Column Volume (CV) or longer. In some embodiments, the first column residence time may be about 0.1 to about 1 minute per Column Volume (CV) or shorter, and the second residence time may be between about 5 minutes to about 10 minutes per Column Volume (CV) or longer.

It is apparent to a skill in the art that the ratio between the first and second column residence time can be determined by a person of skill in the art depending on the nature of the column, the target molecule used and other reaction conditions.

The modulation of flow rate/column residence time may be applied independently of, or in conjunction with addition of an additive, i.e., an N-containing heteroaromatic compound.

### Modulation of pH

In other aspects, it has further been found that modulation of the pH between the intein-complex formation step and the target molecule release/elution step further facilitates target release. Accordingly, any one or more of the methods described above relating to modulation of the flow rate/column residence time and/or addition of an N-heteroaromatic compound, may further be performed in conjunction with modulating the pH between the capture step (i.e., intein complex formation) and the target elution/release step. In some embodiments, the target elution step is performed at a pH that is lower than the intein formation step. In other embodiments, the intein complex formation step and the target elution steps are performed at about the same pH. The buffer used for intein complex-formation is herein referred to as Capture buffer. The buffer used for target molecule elution is herein referred to as Cleavage buffer.

In some embodiments, the target elution step is performed at a pH that is lower than the intein formation step. In accordance with such embodiments, the intein complex-formation step may comprise loading the intein-C tagged target molecule in a saline Capture buffer having a pH of about 8 to about 10. In other embodiments, the intein complex-formation step comprises loading the intein-C tagged target molecule in a saline Capture buffer having a pH of about 9. In some embodiments, the target molecule elution step may comprise releasing and eluting the target molecule in a saline Cleavage buffer having a pH of about 6 to about 8. In other embodiments, the target molecule elution step may comprise releasing and eluting the target molecule in a saline Cleavage buffer having a pH of about 7. In alternative embodiments, the intein complex-formation step may comprise loading the intein-C tagged target molecule in a saline Capture buffer having a pH of about 8 to about 10, and the target molecule elution step comprises releasing and eluting the target molecule in a saline Cleavage buffer having a pH of about 6 to about 8. In other embodiments, the intein complex-formation step comprises loading the intein-C tagged target molecule in a saline Capture buffer having a pH of about 9, and the target molecule elution step comprises releasing and eluting the target molecule in a saline Cleavage buffer having a pH of about 7.

In some embodiments, the target elution step is performed at a pH that is about the same as the intein formation step. In accordance with such embodiments, the intein complex-formation step may comprise loading the intein-C tagged target molecule in a saline Capture buffer having a pH of about 8 to about 10. In other embodiments, the intein complex-formation step comprises loading the intein-C tagged target molecule in a saline Capture buffer having a pH of about 9. In some embodiments, the target molecule elution step may comprise releasing and eluting the target molecule in a saline Cleavage buffer having a pH of about 8 to about 10. In other embodiments, the target molecule elution step may comprise releasing and eluting the target molecule in a saline Cleavage buffer having a pH of about 9. In alternative embodiments, the intein complex-formation step comprises loading the intein-C tagged target molecule in a saline Capture buffer having a pH of about 8 to about 10, and the target molecule elution step comprises releasing and eluting the target molecule in a saline Cleavage buffer having a pH of about 8 to about 10. In other embodiments, the intein complex-formation step comprises loading the intein-C tagged target molecule in a saline Capture buffer having a pH of about 9, and the target molecule elution step comprises releasing and eluting the target molecule in a saline Cleavage buffer having a pH of about 9.

The Capture buffer and Cleavage buffer may be the same buffer system or different buffer systems. The Cleavage buffer may be supplemented with a N-heteroaromatic additives, with or without a change in the pH as described herein.

### Additional Embodiments of the Present Disclosure

It is apparent to a person of skill in the art that each of the methods described herein, i.e., modulation flow rate/column residence time and/or addition of a catalytic agent (N-heteroaromatic) may be performed alone or in combination with each other. Each of these combinations may further optionally be performed by modulating the pH between intein complex formation and target elution steps.

Thus, in some embodiments, the present invention relates to a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of (a) contacting the intein-C tagged target molecule with the intein-N polypeptide, for a first time period sufficient to form the intein complex; and (b) releasing the target molecule from the intein complex by contacting the intein complex with a medium effective to remove the target molecule, for a second time period which is longer than the first time period; wherein step (b) is performed at a pH that is lower than step (a).

In other embodiments, the present invention relates to a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of (a) contacting the intein-C tagged target molecule with the intein-N polypeptide, for a first time period sufficient to form the intein complex; and (b) releasing the target molecule from the intein complex by contacting the intein complex with a medium effective to remove the target molecule, for a second time period which is longer than the first time period; wherein step (b) is performed at about the same pH as step (a).

In other embodiments, the present invention relates to a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of (a) contacting the intein-C tagged target molecule with the intein-N polypeptide, for a first time period sufficient to form the intein complex; and (b) releasing the target molecule from the intein complex by contacting the intein complex with a heteroaromatic compound comprising at least one ring nitrogen atom; wherein step (b) is performed at a pH that is lower than step (a).

In other embodiments, the present invention relates to a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of (a) contacting the intein-C tagged target molecule with the intein-N polypeptide, for a first time period sufficient to form the intein complex; and (b) releasing the target molecule from the intein complex by contacting the intein complex with a heteroaromatic compound comprising at least one ring nitrogen atom; wherein step (b) is performed at about the same pH as step (a).

In other embodiments, the present invention relates to a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of (a) contacting the intein-C tagged target molecule with the intein-N polypeptide, for a first time period sufficient to form the intein complex; and (b) releasing the target molecule from the intein complex by (i) contacting the intein complex with a medium effective to remove the target molecule, for a second time period which is longer than the first time period; and (ii) contacting the intein complex with a heteroaromatic compound comprising at least one ring nitrogen atom; wherein step (b) is performed at a lower pH than step (a).

In other embodiments, the present invention relates to a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of (a) contacting the intein-C tagged target molecule with the intein-N polypeptide, for a first time period sufficient to form the intein complex; and (b) releasing the target molecule from the intein complex by (i) contacting the intein complex with a medium effective to remove the target molecule, for a second time period which is longer than the first time period; and (ii) contacting the intein complex with a heteroaromatic compound comprising at least one ring nitrogen atom; wherein step (b) is performed at about the same pH as step (a).

As contemplated herein, the increase in contact time (e.g., column residence time and/or decrease in flow rate), the presence of the heteroaromatic compound and/or the reduction in pH during the target molecule removal step facilitates the cleavage reaction and increases the yield of the target molecule. In some embodiments, the yield of the target molecule is increased by at least 5% as compared with an equivalent method not employing one or more of the aforementioned process parameters. In other embodiments, the yield of the target molecule is increased by at least 10% as compared with an equivalent method not employing one or more of the aforementioned process parameters. In other embodiments, the yield of the target molecule is increased by at least 15% as compared with an equivalent method not employing one or more of the aforementioned process parameters. In other embodiments, the yield of the target molecule is increased by at least 20% as compared with an equivalent method not employing one or more of the aforementioned process parameters. In other embodiments, the yield of the target molecule is increased by at least 30% as compared with an equivalent method not employing one or more of the aforementioned process parameters. In other embodiments, the yield of the target molecule is increased by at least 40% as compared with an equivalent method not employing one or more of the aforementioned process parameters. In other embodiments, the yield of the target molecule is increased by at least 50% as compared with an equivalent method not employing one or more of the aforementioned process parameters. In other embodiments, the yield of the target molecule is increased by at least 60% as compared with an equivalent method not employing one or more of the aforementioned process parameters. In other embodiments, the yield of the target molecule is increased by at least 70% as compared with an equivalent method not employing one or more of the aforementioned process parameters. In other embodiments, the yield of the target molecule is increased by at least 80% as compared with an equivalent method not employing one or more of the aforementioned process parameters. In other embodiments, the yield of the target molecule is increased by at least 90% as compared with an equivalent method not employing one or more of the aforementioned process parameters.

### Intein-Mediated Protein Purification Methods

In some embodiments, the present methods may be applied for purification of target molecules by chromatographic separation techniques, e.g., column chromatograph. In accordance with this embodiment, the present disclosure provides a method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of:
(a) providing a sample containing the intein-C tagged target molecule;
(b) loading the sample on a column comprising a chromatography resin, the chromatography resin comprising a covalently-linked N-terminal intein polypeptide, under conditions in which the intein-C polypeptide in the fusion protein binds to the intein-N polypeptide in the resin to form the intein complex, wherein the sample is loaded at a first flow rate/first column residence time;
(c) optionally washing the resin containing the intein complex to remove unbound contaminants;
(d) cleaving the target molecule from the intein complex by:
   (i) contacting the intein complex with a medium effective to cleave the target molecule at a second flow rate that is slower than the first flow rate, or a second column residence time that is longer than the first column residence time; and/or
   (ii) contacting the intein complex with a heteroaromatic compound comprising at least one ring nitrogen atom;
(e) regenerating the chromatography resin;
(f) optionally, performing at least one additional purification cycle by repeating steps (a) to (e) at least once; and
(g) optionally, isolating the target molecule.

In some embodiments, steps (b), (c) (if performed), (d) and (e) is independently performed under static incubation or constant flow representing residence times of 0.1-120 min per Column Volume (CV).

In some embodiments, step (d) is performed at about the same pH as step (b) and optional step (c). In other embodiments, steps (b), optional step (c) and step (d) are each performed in a saline buffer having a pH of about 8 to about 10. In other embodiments, steps (b), optional step (c) and step (d) are each performed at a pH of about 9.

The process of the invention can be performed once, i.e., a single purification and regeneration cycle, but is preferably performed multiple times by subjecting the intein-N column to multiple purification and regeneration cycles.

### (i) Affinity Chromatography Matrices Comprising Intein-Ns

The process described herein utilizes intein-N polypeptides as ligands for affinity chromatography. Accordingly, the present invention, in certain embodiments, provides affinity chromatography matrices comprising an intein-N polypeptide attached to a solid support. In a particular embodiment, the solid support is a chromatography resin or chromatography membrane. In one embodiment, the chromatography resin includes a hydrophilic polyvinyl ether base.

In some embodiments, the chromatography resin is polymer based or includes a polymer. In some embodiments, the chromatography resin includes a hydrophilic polyvinyl ether base or a polymethacrylate. In other embodiments, the chromatography resin is formulated on a solid support, wherein the solid support is a bead or a membrane.

Preferably the solid support compromises organic polymers like hydrophilic vinyl ether based polymer, polystyrene, polyether sulfone, polyamide, e.g., nylon, polysaccharides such as, for example, agarose and cellulose, polyacrylate, polymethacrylate, polyacrylamide, polymethacrylamide, polytetrafluoroethylene, polysulfone, polyester, polyvinylidene fluoride, polypropylene, polyethylene, polyvinyl alcohol, polycarbonate, polymer of a fluorocarbon, e.g., poly (tetrafluoroethylene-co-perfluoro(alkyl vinyl ether)), or combinations or copolymers thereof.

In yet other embodiments, the solid support comprises a support of inorganic nature, e.g., silica, zirconium oxide, titanium oxide and alloys thereof. The surface of inorganic matrices is often modified to include suitable reactive groups. In some embodiments, the solid support may, for instance, be based on zirconia, titania or silica in the form of controlled pore glass, which may be modified to either contain reactive groups and/or sustain caustic soaking, to be coupled to ligands.

Exemplary solid support formats include, but are not limited to, a bead (spherical or irregular), a hollow fiber, a solid fiber, a pad, a gel, a membrane, a cassette, a column, a chip, a slide, a plate or a monolith.

Any suitable technique may be used for attaching the intein-N described herein to a support, e.g., a solid support including those well-known in the art and described herein. For example, in some embodiments, the intein-N may be attached to a support via conventional coupling techniques utilizing, e.g., thiol, amino and/or carboxy groups present in the fragment. In some embodiments, the intein-N polypeptide is attached to the chromatography resin through a functional group selected from the group consisting of hydroxyl, thiol, epoxide, amino, carbonyl epoxide and carboxylic acid For example, bisepoxides, epichlorohydrin, CNBr, N-hydroxysuccinimide (NHS) etc., 1,4-Butanediol diglycidyl ether are well-known coupling reagents, and facilitate the chemical coupling of the intein-N fragment to the solid support. Other coupling agents can be used as known in the art. For a review of coupling methods used to this end, see e.g., Immobilized Affinity Ligand Techniques, Hermanson et al., Greg T. Hermanson, A. Krishna Mallia and Paul K. Smith, Academic Press Inc., 1992, the contents of which are hereby incorporated in their entirety. As well known in the field, parameters such as ligand density or substitution level, pore size of the support etc. may be varied to provide a chromatography resin having desired properties.

Choosing the appropriate conditions for coupling a protein ligand to a solid support is well within the capability of the skilled artisan. Suitable buffers for this process include any non-amine containing buffer such as carbonate, bicarbonate, sulfate, phosphate and acetate buffers. The buffers may further include salts which may be in the range of 5 nM-100 mM.

In some embodiments, the reaction is performed at a temperature ranging from 0° C. to 99° C. In certain embodiments the reaction method is practiced at a temperature less than 60° C., less than 40° C., less than 20° C., or less than 10° C. In some embodiments the method of the invention is practiced at a temperature of about 4° C. In other embodiments the method of the invention is practiced at a temperature of 20° C.

### (ii) Preparation of Intein-C Fusion Proteins

In some embodiments of the present disclosure, the intein-C tagged target molecule is prepared by attaching an intein-C polypeptide to a target molecule to obtain a fusion protein, and expressing the fusion protein in an expression system.

Thus, the methods described herein involve the preparation of intein-C tagged target molecule (e.g., a protein). Intein-C tagged molecules can be prepared by attaching an intein-C polypeptide to a target molecule to obtain a fusion protein, and expressing the fusion protein in an expression system. Methods of preparing fusion, or chimeric, proteins are well known in the art including, but not limited to, standard recombinant DNA techniques. For example, DNA fragments coding for different protein sequences (e.g., a C-intein and a target molecule) are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., Current Protocols in Molecular Biology, 1992, the contents of which are incorporated by reference in their entirety). Moreover, many expression vectors are commercially available that already encode a fusion moiety (e.g., a GST moiety, an Fc moiety).

Preferably, the fusion protein is expressed from an encoding nucleic acid in transiently or stably transfected or transformed prokaryotic or eukaryotic host cells or organisms. Common host cells or organisms for expression of recombinant proteins include, for example, Escherichia coli, Corynebacterium glutamicum, Pseudomonas fluorescens, Lactococcus lactis, Pichia pastoris, Saccharomyces cerevisiae, Zea maize, Nicotinia tabacum, Daucus carota, SF9 cells, CHO cells (e.g., CHO DG44 cells, CHO DXB11 cells), NS0 cells, HEK 293 cells, and whole animals such as cows and goats. In an embodiment, the C-intein-target fusion protein is expressed in E. coli. The expressed fusion protein can then be purified away from contaminating cellular proteins using conventional separation and chromatographic methods, such as clarification by depth filtration, purification by anion and cation exchange chromatography, and concentration by ultrafiltration.

In some embodiments, the intein polypeptide (e.g., C-intein) and target protein are linked directly via a peptide bond. In other embodiments, the fusion protein includes a spacer, or linker, molecule between the intein polypeptide (e.g., C-intein) and the target molecule. Suitable spacer/linker molecules are known in the art.

(iii) Affinity Purification

In some embodiments, the intein complex formation step (b) comprises contacting the chromatography resin with a cell culture supernatant comprising the intein-C tagged target molecule. Thus, in some embodiments, this step comprises loading the intein-C tagged target molecule in a saline Capture buffer having a pH of about 8 to about 10, preferably a saline Capture buffer having a pH of about 9.

Conditions under which the C-intein polypeptide in the fusion protein selectively binds to the chromatography bound N-intein polypeptide to form an intein complex can vary depending on the inteins used and can be determined by one of ordinary skill in the art. Exemplary binding conditions include a) a temperature in the range of about 4-25° C., and a buffer comprising 100 mM Tris-HCl, 25 mM NaCl, 0.1 mM zinc chloride, pH=9; b) a temperature in the range of about 4-25° C., and a buffer comprising 50 mM NaAc, 0.5 M NaCl, pH=5; c) a temperature in the range of about 4-25° C., and a buffer comprising 0.5 M NaCl, 10 mM Tris-HCl, pH=8; d) a temperature in the range of about 4-25° C., and a buffer comprising 100 mM Tris, 200 mM NaCl at pH 9; e) a temperature in the range of about 4-25° C., and a buffer comprising 100 mM Tris and 100 mM NaCl at pH 7; and f) a temperature in the range of about 4-25° C., and a buffer comprising 100 mM Tris and 200 mM NaCl at pH 7.

The loaded column may then be optionally washed in step (c) to remove unbound and weakly-bound contaminants using a wash buffer. The washing buffer preferably comprises a detergent (e.g., Triton X100, ND40), a salt (e.g., acetate, phosphate, chloride, sulfate salts of sodium, ammonium, or potassium), a chaotropic agent, preferably urea or arginine, or a combination thereof.

Subsequently, in step (d), the resin is contacted with a Cleavage buffer to effectuate target cleavage and release from the intein complex. As described above, the target release step is preferably conducted at a flow rate that is slower than the flow rate used to form the intein complex. Additionally, as described above, the target release step is preferably conducted at a column residence time that is longer than the column residence time used to form the intein complex. A catalytic agent (e.g., N-heteroaromatic compound such as an azole or azole-containing compound) may further be added during this step to facilitate the target cleavage, release and elution from the column.

Additionally, the pH may be modulated as described above. Thus, the intein complex formation may be performed at a pH of about 9 as described above, and the target elution step may be performed in a saline buffer having a pH of about 6 to about 8 (e.g., 100 mM Tris, 200 mM NaCl, pH=7), so as to release the target molecule from the intein-C polypeptide. The target molecule is then recovered in the eluate.

Alternatively, steps (b), optional step (c) and step (d) may be performed without changing the pH. Thus, the intein complex formation step (b) and optional washing step (c) may be performed at a pH of about 8 to about 10 as described above, and the target elution step may be performed in a saline buffer having a pH of about 8 to about 10. In another embodiment, Steps (b), (c) (if performed) and (d) may all be performed at a pH of about 9. (e.g., 100 mM Tris, 200 mM NaCl at pH 9). The target molecule is then recovered in the eluate.

The column is then regenerated in step (e) as described above, and then optionally washed with water concurrently or subsequently to the regeneration step, and prior to reuse.

The present subject matter described herein will be illustrated more specifically by the following non-limiting examples, it being understood that changes and variations can be made therein without deviating from the scope and the spirit of the disclosure as hereinafter claimed. It is also understood that various theories as to why the disclosure works are not intended to be limiting.

### EXAMPLES

The following are examples that illustrate embodiments for practicing the disclosure described herein. These examples should not be construed as limiting.

### Example 1: Materials and Methods

Expression of intein-fused protein genes in E. Coli

Intein-C targets were produced in a bioreactor batch, growth conditions: 3 h, 30° C. Intein-N ligands were produced under growth conditions: 20 h, 20° C., flask format. 100 mL 2xYT medium (Merck kGaA) were inoculated with 100 μl kanamycin stock solution (30 mg/ml) and 2 mL of a pre-culture. The main-cultures were grown at 20-30° C. and 200-500 rpm. Induction at 0.12 mM Isopropyl β-D-1-thiogalactopyranoside (IPTG) end concentration took place at an OD600 value of 2. The main cultures were cultivated for 3 or 20 hours. Cells were harvested by centrifugation, the supernatant was discarded, and pellets were stored below −20° C.

Cell Lysis of intein-fused protein expressed E. Coli cells

Biomass was lysed by chemical or mechanical cell lysis. Intein-C tagged (IC) targets were lysed by mechanical cell lysis while chemical cell lysis was used for intein-N (IN) ligands.

Mechanical cell disruption was carried out by suspending cells in 10 mL Mechanical Lysis Buffer (100 mM Tris, 150 mM NaCl, 5 mM MgCl₂ and 25 U/ml Benzonase®, pH 8-9). The cell solution was transferred into a cell disruption chamber and cell disruption was accomplished at 1 kbar. The supernatant (lysate) was centrifuged at 4° C., 18000 rcf for 25 minutes. After the centrifugation, the supernatant was filtered and used as clarified E. Coli cell lysate (CL) for further purification step (e.g., intein-C target E. Coli lysate below).

Chemical cell lysis was carried out by suspending cells in, 10 mL Chemical Lysis Buffer (50 mM Tris, 5 mM MgCl2, 1:10 CelLytic B cell lysis Reagent, 25 U/mL Benzonase®, adjust to pH 8 with HCl). The mix was vortexed and centrifuged. After the centrifugation, the supernatant was filtered was used as clarified E. Coli cell lysate for further purification step.

### Expression and Feed Preparation: Conditioning of Mammalian Intein-C Tagged Target Expressing Cell Lysates

Highly glycosylated intein-C tagged human derived target molecules were produced in HEK293 cells or in CHOZN® GS−/− cell line. The secreted and processed intein-C tagged target molecules within clarified cell supernatant was concentrated using a Pellicon XL 5 kDa membrane to adjust a concentration of −0.1-0.3 mg/ml intein-C tagged target molecule. The concentration was determined using SDS-Page Analysis of the sample. The concentrated supernatant was conditioned to adjust to pH 9 using 2 M NaOH and loaded to an intein-N ligand prototype column (e.g. clarified mammalian cell supernatant below).

### StrepII-Tag Based Purification Method

An affinity column packed with Strep-Tactin® Superflow HC was equilibrated with 2 column volume (CV) Strep-Binding Buffer (100 mM tris, 200 mM NaCl, pH 9 for intein-C targets; and 100 mM Tris, 150 mM NaCl and 1 mM EDTA, pH 8 for intein-N ligands). The clarified E. Coli cell lysate was loaded to the column, unbound protein was washed through the column with Strep-Binding Buffer and bound target was eluted with Strep-Elution Buffer (100 mM Tris, 200 mM NaCl and 2.5 mM d-Desthiobiotin, pH 9 for intein-C targets; and 100 mM Tris, 150 mM NaCl, 1 mM EDTA, 1 mM TCEP and 2.5 mM d-Desthiobiotin, pH 8 for intein-N ligands). The column was regenerated by eluting of remaining protein with Strep-Regeneration Buffer (50 mM Tris, 150 mM NaCl, 1 mM 4″-hydroxyazobenzene-2-carboxylic acid (HABA), 1 mM EDTA, pH 8). After another column wash with 100 mM Tris Buffer, the column was re-equilibrated with 2CV in Strep-Binding Buffer.

### Dialysis and Intein-N/Resin Coupling Reaction

StrepII-tagged and purified intein-N ligand was injected into Dialysis Cassettes. Cassettes were transferred into a beaker containing Coupling Buffer (100 mM Na₂CO₃/NaH₂—CO₃, 1 mM TCEP, pH 10). Dialysis was performed at 4° C. overnight. Dried Epoxy modified Eshmuno® resin was swollen using 2 mL of Coupling Buffer without reducing agents to achieve a 1 mL column size. The swelled resin was sucked dry. Dialysed intein-N ligand was then transferred to the swelled resin. The resin was incubated in a 1:3 relation (v/v) to the intein-N ligand Stock for 2.5 hr. The resin was quenched using Quenching Buffer (100 mM Na₂CO₃/NaH₂CO₃, 0.1-1 M glycine, pH 8).

Production of proteins of the expected size was confirmed using SDS polyacrylamide electrophoresis (SDS PAGE) as known in the art. The amount of covalently bound ligand was determined through a BCA assay as known in the art.

### Functionality Test: Dynamic Binding Capacity in Column Format

For testing intein-N resin prototypes according to its performance in dynamic column process, the intein purification method was conducted with intein-N resin prototype columns. A sample containing 50% (v/v) resin bulk was transferring to a Scout column with a final resin column volume of 1 mL. The packed prototype column was then used for intein-based purification method.

### Purification Method

The resin was equilibrated with 10 CL Capture Buffer (100 mM Tris, 200 mM NaCl, pH=9). A sample size of 5CV of an intein-C target solution that was pre-purified using the StrepII-Tag purification method described above, or an intein-C tagged target containing clarified mammalian cell lysate, conditioned as described above was loaded to the column. The column was washed with 10 CV Capture Buffer and bound intein-C target was released triggered by a pH reduction with 10 CV Cleavage Buffer (100 mM Tris, 200 mM NaCl, pH=7). The column was then cleaned from remaining intein-C fragments using 5 CV CIP buffer (150 mM H₃PO₄ (pH 1-2)). The column was then re-equilibrated with 5 CV Capture Buffer.

### Purified Sample Analysis.

Before and after the StrepII-Tag or intein-tag based purification step, the A280-Absorbance (Absorbance at 280 nm wavelength) was calculated for all fractions with the chromatography software to check the amount of protein in each fraction. Under consideration of the A280-Absorbance and the extinction coefficient of the target, the concentration of the eluate and CIP fractions were determined.

Samples were analyzed using SDS gel electrophoresis (SDS PAGE) and capillary electrophoresis (LapChip®) as known in the art. For LabChip® analysis, samples were heated with Denaturation LabChip® Buffer enriched with DTT for 5 minutes. The samples were filled up with distilled H₂O to 35 μl and was separated and analyzed using a Protein Express Assay.

### Example 2: Increased Intein Cleavage at Standard Cleavage Conditions (pH 7) with Imidazole, Pyrazole and Histidine

An intein-N resin prototype column carrying a third generation of intein-N ligand (R44-358132) was used for five intein purification cycles (Example 2-1: Cycles 1.1-1.5) using different additives at different concentrations in a standard cleavage condition buffer system (pH 7). The purification was conducted with 13 kDa intein-C tagged target molecule.

A second intein-N resin prototype column (carrying R46-358132) was used for five consecutive intein purification cycles (Example 2-2: Cycles 2.1-2.5) with another target molecule (36 kDa) and another resin batch. The two intein-C tagged targets (13 and 36 kDa) (corresponding to a fused target molecule named Thioredoxin (UniProtKB—POAA25 (THIO_ECOLI), MW=13 kDa and a target molecule named Curved DNA-binding protein (UniProtKB—P36659 (CBPA_ECOLI) MW=36 kDa respectively). Both proteins are from the proteome of the organism Escherichia coli (strain K12), were pre-purified using Strep-Tag® purification as described in Example 1 and were diluted to a concentration of 1 mg/mL. The intein-N immobilized resin was equilibrated with 10 CV Capture Buffer (100 mM Tris, 200 mM NaCl, pH 9) and one of the pre-purified intein-C targets was loaded with 5 CV to the intein-N resin prototype column under capture conditions (100 mM Tris, 200 mM NaCl, pH 9). The unbound proteins were washed out with 10 CV Capture Buffer. The standard Cleavage Buffer (100 mM Tris, 200 mM NaCl, pH 7) (B.1) was enriched with 0.3 M imidazole (B.2), 0.6 M imidazole (B.3), 0.3 M histidine (B.4) or 0.3 M pyrazole (B.5) as described in Table 1:

TABLE 1
Cleavage Buffer with different Additives

| Buffer | Concentration | Substance |
|---|---|---|
| Cleavage Buffer pH 7 | — | — |
| B.1 | | |
| Cleavage Buffer pH 7 | 300 mM | Imidazole |
| B.2 | | |
| Cleavage Buffer pH 7 | 600 mM | Imidazole |
| B.3 | | |
| Cleavage Buffer pH 7 | 300 mM | Histidine |
| B.4 | | |
| Cleavage Buffer pH 7 | 300 mM | Pyrazole |
| B.5 | | |

Adjust to pH 7 with HCl

The intein cleavage reaction and tagless target release was triggered by a pH shift step to a lower pH value using one of the given Cleavage Buffers B.1-B.5 and the elution was accomplished in a two-step approach. The first elution was triggered under dynamic flow with 4 CV Cleavage Buffer B.1-B.5. The second elution was triggered with 6 CV Cleavage Buffer B.1-B.5 after setting the flow on hold to achieve a 2 h static column incubation. The chromatography column was regenerated using at least 5 CV acidic solutions with pH between 1-2 containing for example 0.15 M H₃PO₄ or similar buffer and was reused for the next cycle of intein purification.

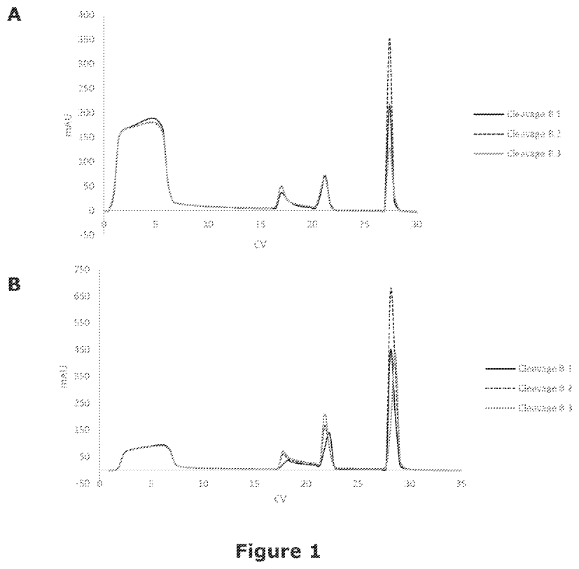

Five different Cleavage Buffers B.1-B.5 were used in five consecutive purification cycles. Each run was performed with the same intein-C target protein and the same column prototype. For analysis, the A280-Absorbance chromatograms were analyzed according to the protein amounts in the elution and CIP fractions using the appropriate extinction coefficient of the target. Two overlays of the chromatograms demonstrate the different elution behavior during cycle 1.1, 1.2 and 1.3 (FIG. 1A) and the elution of the 13 kDa-target molecules as well as during cycle 2.1, 2.2 and 2.3 (FIG. 1B) and the elution of the 36 kDa target molecule. Cycles 1.4, 1.5, 2.4 and 2.5 are not shown due to background absorption at 280 nm (A280-Absorbance) of a histidine- and pyrazole-containing tris buffer system. As shown, the addition of 0.3 M (B.2) or 0.6 M (B.3) imidazole resulted in a higher amount of target elution that was recovered from elution fractions 1 and 2, as compared with the reference buffer (B.1).

The amount of eluted target during elution step 1 and step 2 was calculated for all ten cycles using the A280-Absorbance and the molecular extinction coefficient of the cleaved target and is listed in Tables 2 and 3. The amount of elution of the 13 kDa tagless target was about 0.26-0.33 mg and about 0.58-0.73 mg for the 36 kDa target, depending on the used Cleavage Buffer B.1-B.5). Due to the cleavage activity of the intein-C target stock, some uncleaved intein-C target remained on the column after the elution phase. The total intein-C bound protein was calculated using the eluted target and the remaining intein-C target, that was released during the cleaning phase together with the remaining intein-C fragments. The yield of tagless released target during the elution phase was calculated per total bound intein-C target. The yield was calculated to be above 41.79% for both targets (Tables 2 and 3). Using both prototype column, the total bound amount of intein-C tagged target stays consistent over several purification cycles using both Intein-C tagged targets.

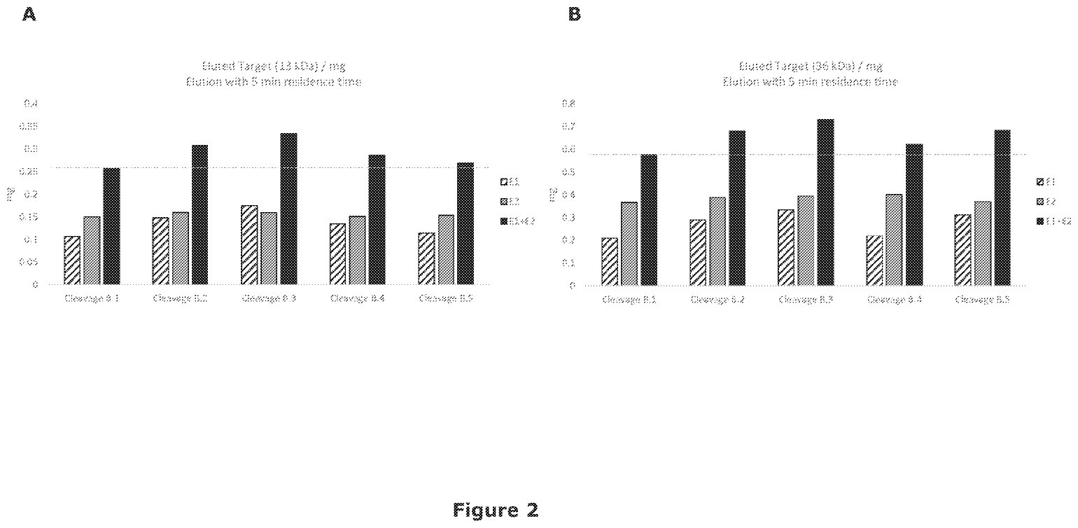

The positive effect of azole- and azole-containing compounds on the intein functionality was verified with both target proteins. The elution yield was increased up to 9.87% (Table 2) or 4.01% (Table 3), whereas the amount of cleaved target that was collected during elution step 1 (E1) could be increased up to 11.15% (Table 2) or 9.72% (Table 3), using one of the imidazole or imidazole-derivate containing Cleavage Buffers (B.2-B.5, FIG. 2A, 2B) within Example 2-1, Table 2 (using the 13 kDa target) or Example 2-2, Table 3 (using the 36 kDa target).

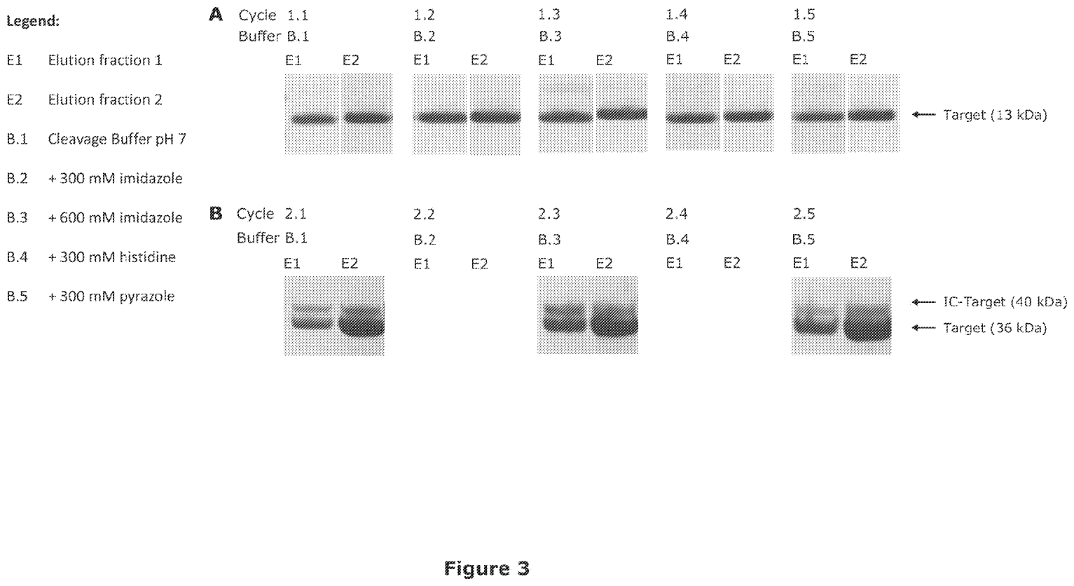

A sample of the elution fraction (E1, E2) was analyzed by SDS-PAGE gel electrophoresis as shown in FIG. 3. In both Examples 2-1 and 2-2, the tagless released target could be observed (13 kDa in 2-1 and 36 kDa in 2-2), when elution was triggered with the different elution conditions (B.1-B.5). Due to overloading effects of the column, some uncleaved intein-C target was observed in example 2-2 as well.

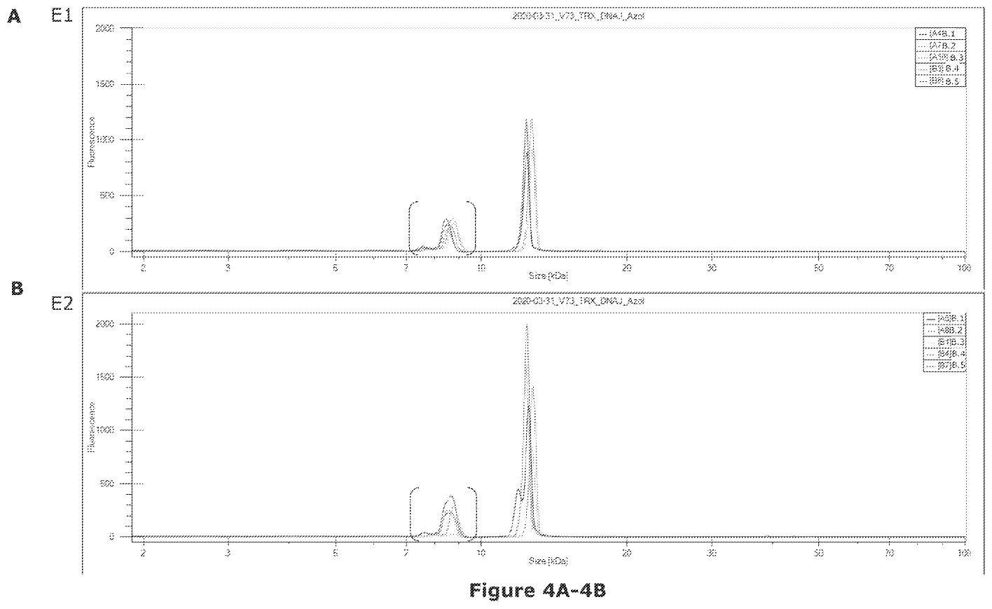

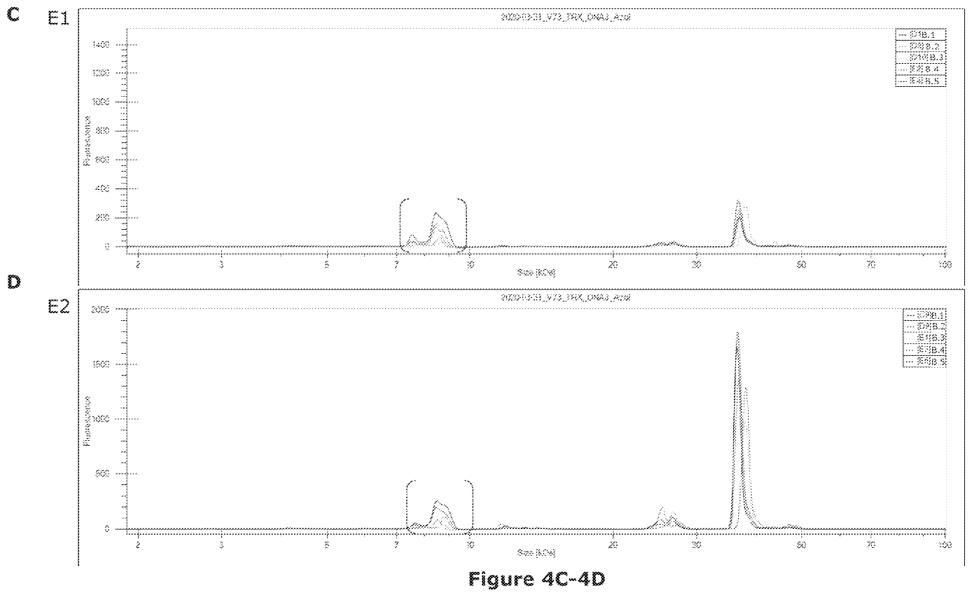

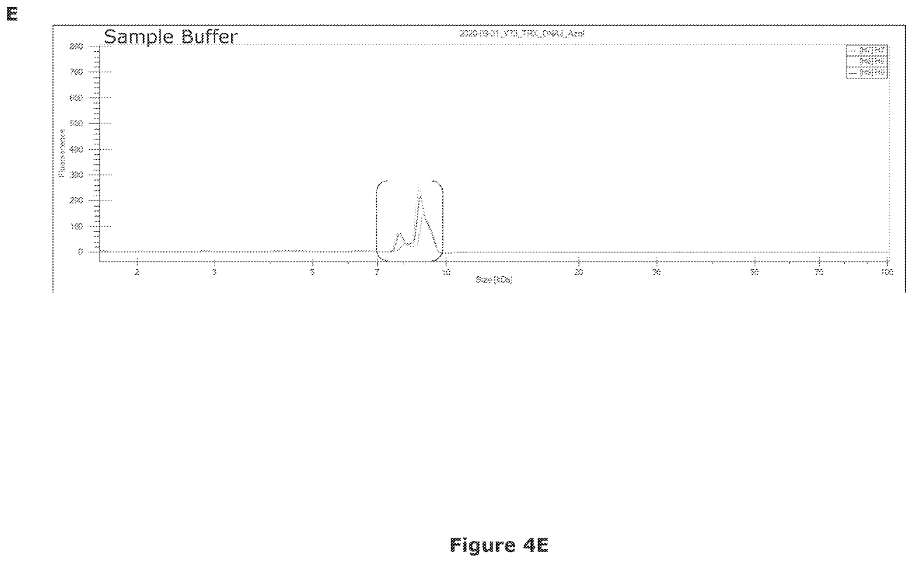

The purity was calculated from the elution fractions using a LabChip®GXII™ microfluidic electrophoretic separation system in combination with a Protein Express Assay Reagent Kit and a LabChip® HT Protein Express Chip. The purity of the eluted target was calculated to be consistent above 97.42% for the 13 kDa target. The 36 kDa target that was used in Example 2-2 resulted in a lower amount of protein release and a lower purity level of the elution fractions (above 73.04% purity). The values are listed in Table 2 and Table 3. The overlay in FIG. 4 shows the purity of all elution fractions of cycle 1.1-1.5 as well as 2.1-2.5, analyzed by microfluidic electrophorese system. FIG. 4A, 4B (Example 2-1) shows two overlays of all E1 (FIG. 4A) and E2 (FIG. 4B) fractions, collected during the intein purification cycles 1.1-1.5. FIG. 4C, 4D (Example 2-2) shows two overlays of all collected E1 (FIG. 4C) and E2 (FIG. 4D) fractions, collected during the intein purification cycles 2.1-2.5. The marked double-peak between 7 and 9 kDa was excluded from purity calculation due to unspecific background noise of the Denaturation Sample Buffer. FIG. 4E shows the LabChip® Sample Buffer without proteins, for demonstrating the unspecific background noise, exemplary for the whole study.

### Results Summarized in Tables 2 and 3

Table 2 shows the results of Example 2-1 with an intein-N resin prototype column. The percentage of eluted target during 5 consecutive cycles of intein purification was calculated using the total protein amount recovered from elution (E1+E2) and the regeneration fractions (CIP: remaining intein-C target and intein-C fragment (IC)). The amount of eluted target was compared to the elution under standard conditions (B.1). As shown, a 6.56% and 7.79% higher yield of target was recovered using imidazole containing elution buffer (B.2, B.3) during purification cycle 1.2 and 1.3. The effect of increased elution yield was also observed in following purification cycle 1.4 and 1.5, here a 2.17% and 9.87% yield improvement could be calculated. Especially the yield of cleaved target during the elution step 1 (E1) per total cleaved intein-C target could be increased up to 11.15% (cycle 1.3). The yield was calculated using the target recovered from elution and the total bound intein-C target to the column. All elution fractions except E2-fraction of cycle 1.2 contained nearly 97-100% pure tagless target.

TABLE 2
Example 2-1

| Purification Cycle | 1.1 | 1.2 | 1.3 | 1.4 | 1.5 |
|---|---|---|---|---|---|
| Elution Buffer | Cleavage B.1 | Cleavage B.2 | Cleavage B.3 | Cleavage B.4 | Cleavage B.5 |
| Target | 13 kDa | 13 kDa | 13 kDa | 13 kDa | 13 kDa |
| Ligand | R44- | R44- | R44- | R44- | R44- |
| | 358132 | 358132 | 358132 | 358132 | 358132 |
| Total bound intein-C | 0.53 | 0.56 | 0.59 | 0.56 | 0.46 |
| target/mg | | | | | |
| E1/mg | 0.11 | 0.15 | 0.18 | 0.14 | 0.12 |
| E2/mg | 0.15 | 0.16 | 0.16 | 0.15 | 0.15 |
| E1 + E2/mg | 0.26 | 0.31 | 0.33 | 0.29 | 0.27 |
| Yield of cleaved | 48.68% | 55.24% | 56.47% | 50.85% | 58.55% |
| target per total | | | | | |
| bound IC-target | | | | | |
| % improvement in | — | 6.56% | 7.79% | 2.17% | 9.87% |
| yield (total) | | | | | |
| Yield of cleaved | 41.25% | 48.05% | 52.40% | 47.20% | 42.75% |
| target in E1 per total | | | | | |
| cleaved IC-target | | | | | |
| % improvement in | — | 6.80% | 11.15% | 5.95% | 1.50% |
| yield (E1) | | | | | |

Calculations from LabChip ® Assay

| | | | | | |
|---|---|---|---|---|---|
| E1 Purity/% | 100.00 | 100.00 | 98.68 | 99.07 | 97.47 |
| E2 Purity/% | 98.02 | 83.82 | 100.00 | 100.00 | 97.42 |

Table 3 shows the results of Example 2-2 with an intein-N resin prototype column. The percentage of eluted target during 5 consecutive cycles of intein purification was calculated using the total protein amount recovered from elution (E1+E2) and the regeneration (CIP: remaining intein-C target and intein-C fragment (IC)). The amount of eluted target was compared to the elution under standard conditions (B.1). Thus, a 0.70% and 4.01% higher yield of target was recovered using imidazole containing elution buffer (B.2, B.3) during purification cycle 2.2 and 2.3. The effect of increased elution yield was also observed in following purification cycle 2.5, here a 3.83% yield improvement was calculated. Especially the yield of cleaved target during the elution step 1 per total cleaved intein-C target was increased up to 9.72% (cycles 2.3, 2.5). The yield was calculated using the target recovered from elution and the total bound intein-C target to the column. All purities of the elution 1 fractions were lower than the purities of the elution 2, but in a consistent level of 70-80%.

TABLE 3
Example 2-2

| Purification Cycle | 2.1 | 2.2 | 2.3 | 2.4 | 2.5 |
|---|---|---|---|---|---|
| Elution Buffer | Cleavage B.1 | Cleavage B.2 | Cleavage B.3 | Cleavage B.4 | Cleavage B.5 |
| Target (MW) | 36 kDa | 36 kDa | 36 kDa | 36 kDa | 36 kDa |
| Ligand | R46- | R46- | R46- | R46- | R46- |
| | 358132 | 358132 | 358132 | 358132 | 358132 |
| Total bound intein-C | 1.24 | 1.44 | 1.45 | 1.49 | 1.36 |
| target/mg | | | | | |
| E1/mg | 0.21 | 0.29 | 0.34 | 0.22 | 0.31 |
| E2/mg | 0.37 | 0.39 | 0.40 | 0.40 | 0.37 |
| E1 + E2/mg | 0.58 | 0.68 | 0.73 | 0.62 | 0.68 |
| Yield of cleaved | 46.59% | 47.29% | 50.60% | 41.79% | 50.42% |
| target per total bound | | | | | |
| IC-target | | | | | |
| % improvement in | — | 0.70% | 4.01% | −4.80% | 3.83% |
| yield (total) | | | | | |
| Yield of cleaved | 36.11% | 42.79% | 45.83% | 35.53% | 45.83% |
| target in E1 per total | | | | | |
| cleaved IC-target | | | | | |
| % improvement in | — | 6.68% | 9.72% | −0.58% | 9.72% |
| yield (E1) | | | | | |

Calculations from LabChip ® Assay

| | | | | | |
|---|---|---|---|---|---|
| E1 Purity/% | 79.48 | 73.04 | 73.63 | 79.54 | 81.59 |
| E2 Purity/% | 88.45 | 89.61 | 88.66 | 80.97 | 90.45 |

### Example 3: Verification of Azole and Azole-Derived Effects on Intein Cleavage Process Under Capture Conditions (pH 9)

An intein-N resin prototype column carrying a third generation of intein-N ligand (R44-358132) was used for two intein purification cycles studying the improvement of intein cleavage in a standard capture condition buffer system (pH 9) using azole and azole-derived additives showed positive effects on intein functionality (Example 2). A 13 kDa intein-C tagged target was pre-purified using Strep-II Tag purification as described in example 1 and was diluted to a concentration of 1 mg/mL. The intein-N resin prototype column was equilibrated with 10 CV Capture Buffer (100 mM Tris, 200 mM NaCl, pH 9). The pre-purified intein-C target was loaded with 5 CV to the prototype column under capture conditions (100 mM Tris, 200 mM NaCl, pH 9). The impurities were washed out with 10 CV Capture Buffer (100 mM Tris, 200 mM NaCl, pH 9). The Capture Buffer designated A.1 (100 mM Tris, 200 mM NaCl, pH 9) was used as a reference in this setup. To study the effect of catalytic effect of additive addition, the conditions of the reference buffer were adjusted with 0.3 M imidazole (Capture Buffer A.2). The intein cleavage reaction and tagless target release was triggered and accomplished in a two-step approach. The first elution was triggered under dynamic flow with 4CV Capture Buffer A.1-A.4 as described in Table 4. The second elution was triggered with 6CV Capture Buffer A.1 and A.2 after putting the flow on hold to achieve a 2 h static column incubation. The chromatography column was regenerated using at least 5 CV acidic solutions with pH between 1-2 containing for example 0.15 M H₃PO₄ and the column was reused for the next cycle of intein purification.

TABLE 4
Capture Buffer with different Additives

| Buffer | Concentration | Substance |
|---|---|---|
| Capture Buffer pH 9 | — | — |
| A.1 | | |
| Capture Buffer pH 9 | 300 mM | Imidazole |
| A.2 | | |

Adjust pH 9 with HCl and NaOH

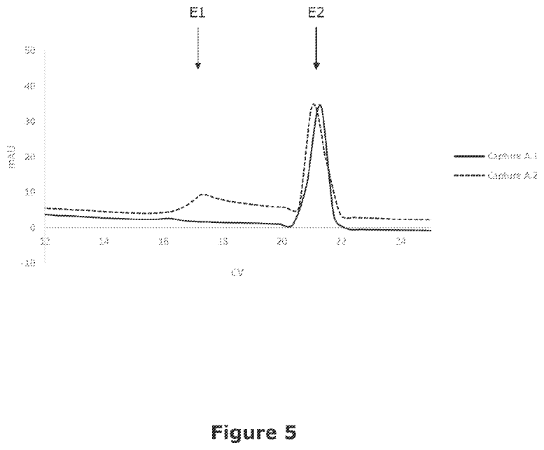

The two different Capture Buffers A.1 and A.2 were used in two consecutive purification cycles. The A280-Absorbance chromatograms were recorded and analyzed to determine the amount of protein recovered from the elution and the CIP phase. To demonstrate the catalytic effect of additive addition, the A280-Absorbance chromatograms of purification cycle 1.1 and 1.2 were compared in an overlay (FIG. 5).

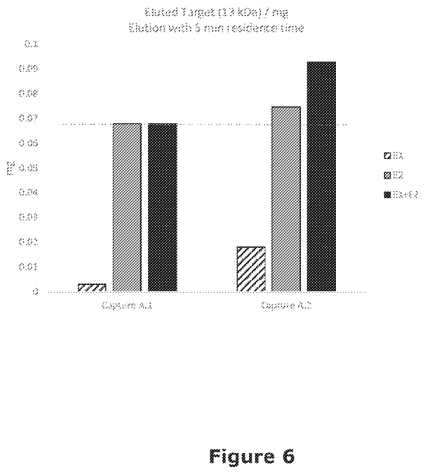

The amount of eluted target recovered from the elution step 1 and elution step 2 were determined using the A280-Absorbance and the molecular extinction coefficient of the cleaved target. The total bound amount of intein-C tagged protein was calculated using the eluted target, and the remaining intein-C target, that was released during the cleaning phase together with the remained intein-C fragments. The yield of tagless released target during the elution phase was calculated per total bound intein-C target. The eluted 13 kDa tagless target yield was calculated to be 10.91% of the total bound intein-C target under capture conditions (yield), whereas 13.88% of the total bound protein was eluted using imidazole-enriched Capture Buffer (A.2). The yield of cleaved target that was collected during the elution step 1 (E1) per total eluted cleaved target was increased from 4.20% to 19.35%, using imidazole-enriched Capture Buffer (A.2) (Table 5 and FIG. 6).

This example demonstrates that the positive effect of imidazole on the intein functionality and especially on the kinetic of the reaction could be verified for an intein purification process while introducing a pH shift during the elution phase (Example 2, up to 11.15% increased elution yield during elution step 1) as well as without introducing a pH shift (Example 3, 15.15% increase in elution yield during elution step 1 (E1)).

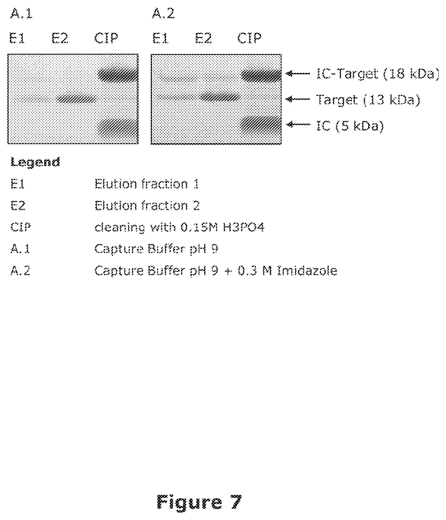

A sample of the elution (E1, E2) was analyzed by SDS-PAGE gel electrophoresis as shown in FIG. 7.

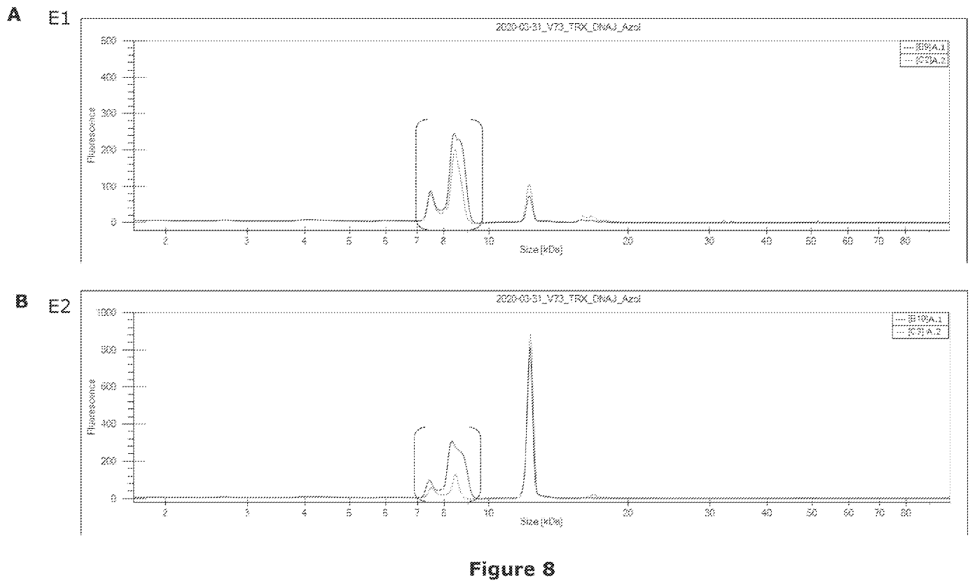

The purity was calculated from the elution fractions using a LabChip®GXII™ microfluidic electrophoretic separation system in combination with a Protein Express Assay Reagent Kit and a LabChip® HT Protein Express Chip. The calculated purity of the eluted target was very low in the elution fraction 1 but higher in the elution fraction 2. The longer the incubation time of the column at pH 9, the higher the amount of protein and purity that was measured in elution fraction 2. The amount and purity level in the setup with imidazole showed in the elution 1 as well as in the elution 2 a constant purity above 62.66%. Recorded purities are listed in Table 5. The overlay in FIG. 8 shows the purity of all elution fractions.

Table 5 shows the elution cycle study with an intein-N resin prototype column. The percentage of eluted target recovered from two consecutive purification cycles was calculated using the total protein amount recovered from the elution fraction (E1+E2) and the regeneration fraction (CIP: remaining intein-C target and intein-C fragment (IC)). The amount and yield of eluted protein for imidazole containing buffer A.2 (Table 4) was compared against the amount and yield of eluted protein under reference conditions (Capture Buffer A.1— Table 4). The yield of 10.91-13.88% was calculated using the target recovered from elution and the total bound intein-C target. The purity was calculated to be lower using the imidazole-enriched Capture Buffer.

TABLE 5

| Purifcation Cycle | 1 | 2 |
|---|---|---|
| Elution Buffer | Capture A.1 | Capture A.2 |
| Target | 13 kDa | 13 kDa |
| Ligand | R44-358132 | R44-358132 |
| Total bound intein-C target/mg | 0.65 | 0.67 |
| E1/mg | 0.003 | 0.02 |
| E2/mg | 0.07 | 0.08 |
| E1 + E2/mg | 0.07 | 0.09 |
| Yield of cleaved target per total bound | 10.91% | 13.88% |
| IC-target | | |
| % improvement in yield (total) | — | 2.97% |
| Yield of cleaved target in E1 per total | 4.20% | 19.35% |
| cleaved IC-target | | |
| % improvement in yield (E1) | — | 15.15% |

Calculations from LabChip ® Assay

| | | |
|---|---|---|
| El Purity/% | 98.00 | 66.84 |
| E2 Purity/% | 98.00 | 62.66 |

### Example 4: Azole Triggered Intein Cleavage Process with Different Residence Times During Elution of Two Target Molecules with and without Introducing a pH Shift

The intein-N resin prototype column, carrying a third generation of intein-N ligand (R46-358132) was used for five intein purifications. The improvement of intein cleavage through additives such as imidazole (Examples 2, 3) was studied under longer residence times, with and without introducing a pH shift during the elution phase.

One reference run (cycle 1) was accomplished as a reference run for that study, using 5 minutes residence time during the whole purification process (0.2 ml/min flow rate). Further, the intein-N resin prototype column was used in additional four consecutive cycles (cycles 2-5) where a combinatorial method flow rate was given. The residence time of the elution was set at 10 minutes while using four different elution buffers (Cleavage Buffer B.1 and B.2 as well as Capture Buffer A.1 and A.2). The other process steps within cycle 2-5 were accomplished with a 1 ml/min flow rate (1 minute residence time). A 13 kDa intein-C tagged target was pre-purified using Strep-Tag® purification and was diluted to a concentration of 1 mg/mL. The intein-N resin prototype column was equilibrated with 10 CV Capture Buffer (100 mM Tris, 200 mM NaCl, pH 9) and the pre-purified intein-C target was loaded with 5 CV to the prototype column. The loading was accomplished under capture conditions (100 mM Tris, 200 mM NaCl, pH 9). The impurities were washed out with 10 CV Capture Buffer (100 mM Tris, 200 mM NaCl, pH 9). The elution of the 13 kDa target molecule was activated by a pH reduction step to a lower pH level using one of the given Cleavage Buffers B.1 and B.2. The standard Cleavage Buffer, used for cycle 1 and 2, was Buffer B.1 (100 mM Tris, 200 mM NaCl, pH 7). This buffer was enriched with 0.3 M imidazole (B.2) and used for purification cycle 3.

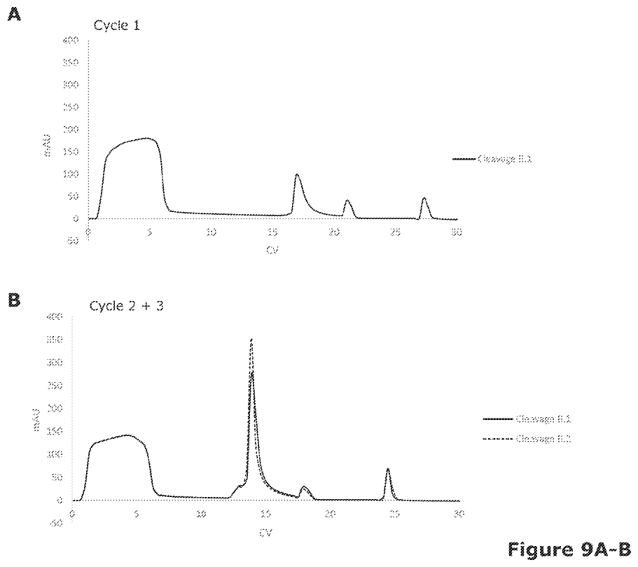

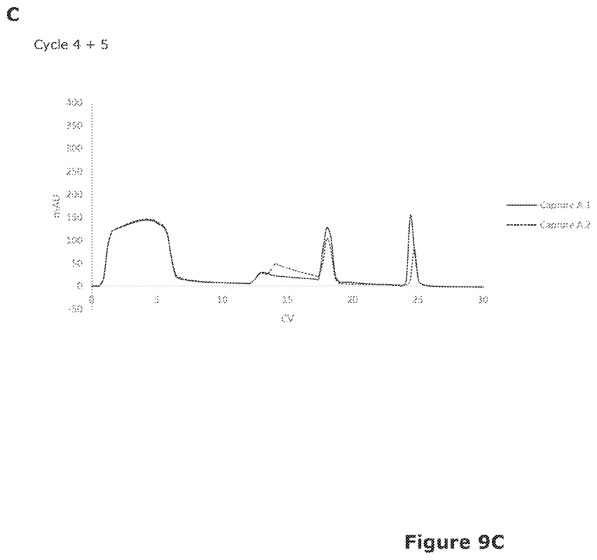

The elution of the 13 kDa target of the next two rounds of intein purification (cycle 4 and 5) were done with the Capture Buffer A.1 (100 mM Tris, 200 mM NaCl, pH 9) and the imidazole-enriched Capture Buffer A.2 (0.3 M imidazole) having the same pH, implying that no pH level reduction in the elution phase was given. The consecutive elutions of the described five purification cycles were performed in a two-step approach. The first elution was triggered under dynamic flow with 6CV Cleavage Buffer B.1 and B.2 as well as Capture Buffer A.1 and A.2. The second elution was triggered with 4CV Cleavage Buffer B.1 and B.2 as well as Capture Buffer A.1 and A.2 after putting the flow of the column on hold to achieve a 2 h static column incubation. The chromatography columns were regenerated using at least 5 CV acidic solutions with pH between 1-2 containing for example 0.15 M H₃PO₄ and was reused for the next round of intein purification. Two chromatogram overlays demonstrate the different elution behavior during cycle 1-5 (FIG. 9). As shown, the addition of 0.3 M imidazole resulted in a higher elution amount during elution 1 and 2.

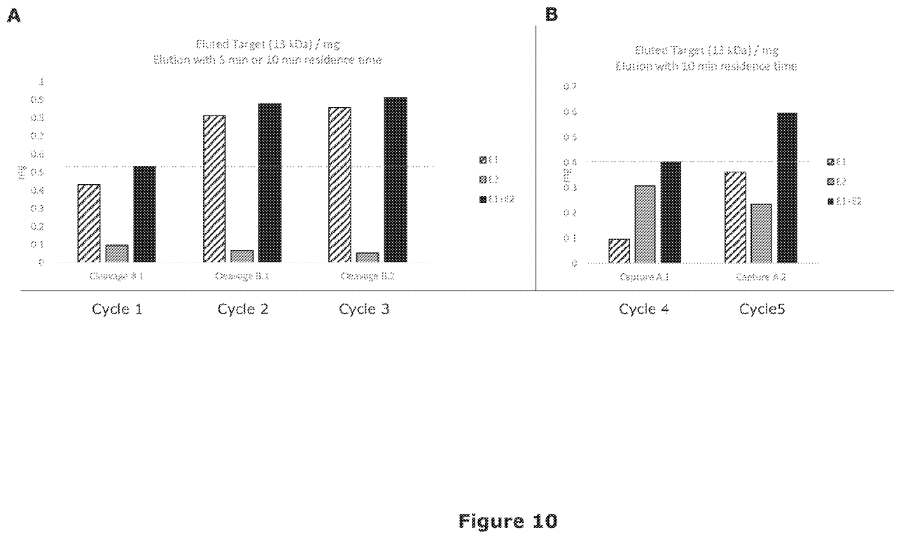

Depending on the used buffer system during the elution phase, the amount of eluted tagless 13 kDa target was about 0.40-0.91 mg. Due to the cleavage activity of the intein-C target stock, some uncleaved intein-C target remained on the column after the elution phase. The total intein-C bound protein was calculated using the eluted target and the remaining intein-C target, that was released during the cleaning phase together with the remained intein-C fragments. The amount of eluted target during elution step 1 and step 2 was calculated for all five cycles using the A280-Absorbance and the molecular extinction coefficient of the cleaved target. The amounts are listed in Table 6. Just by increasing the residence time by reducing the flow rate from 0.2 ml/min to 0.1 ml/min, the elution yield was increased from 94.80% to 97.79% and the yield of cleaved target that was collected during the first elution step (E1) was increased from 81.57% to 92.38% and to 94.08% by adding imidazole-rich buffer (Cleavage Buffer B.1.). The yield that was calculated from purification cycle 5 was increased by 23.31%, compared to cycle 4 whereas the target release during the first elution step (E1) was increased by 36.88% using imidazole-enriched Capture Buffer A.2 (cycle 5) instead of Capture buffer A.1 (cycle 4, FIG. 10).

The yield of tagless released target during the elution phase was calculated per total bound amount of intein-C target. The yield was calculated to be >94.80% or 66.02-89.33% using Cleavage Buffer B.1/B.2 or Capture Buffer A.1/A.2 as an elution buffer, respectively (Table 6). The enrichment of imidazole resulted in a higher yield of recovered target during the elution phase using both pH-conditions.

When comparing the process yields of Example 4 to Example 3 and 2, it is apparent that the achieved process yields between the examples vary between 10.91-66.02% using capture conditions for triggering the elution and 48.68%-94.80% using a pH shift to pH 7 during the elution.

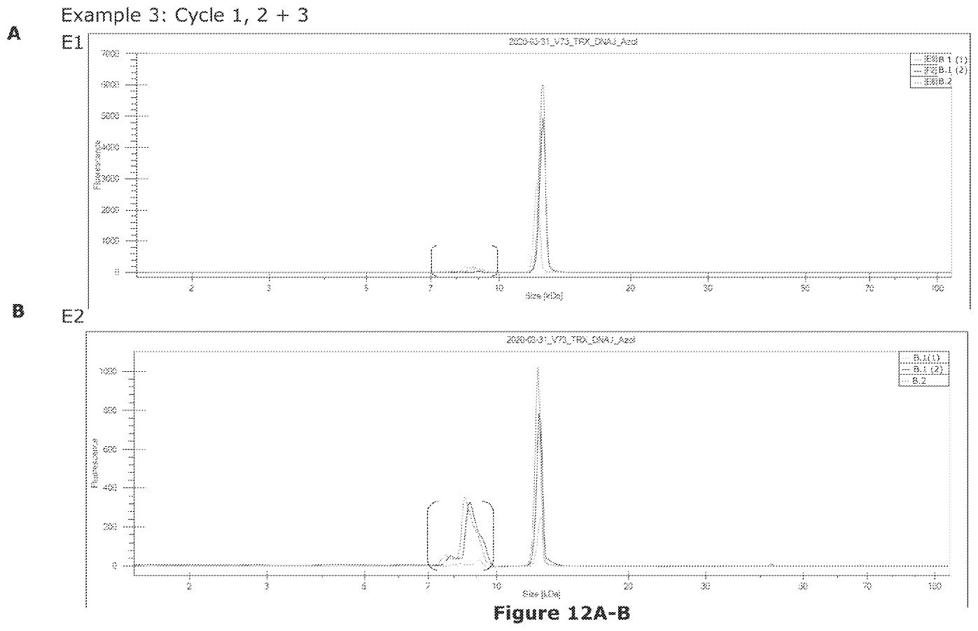

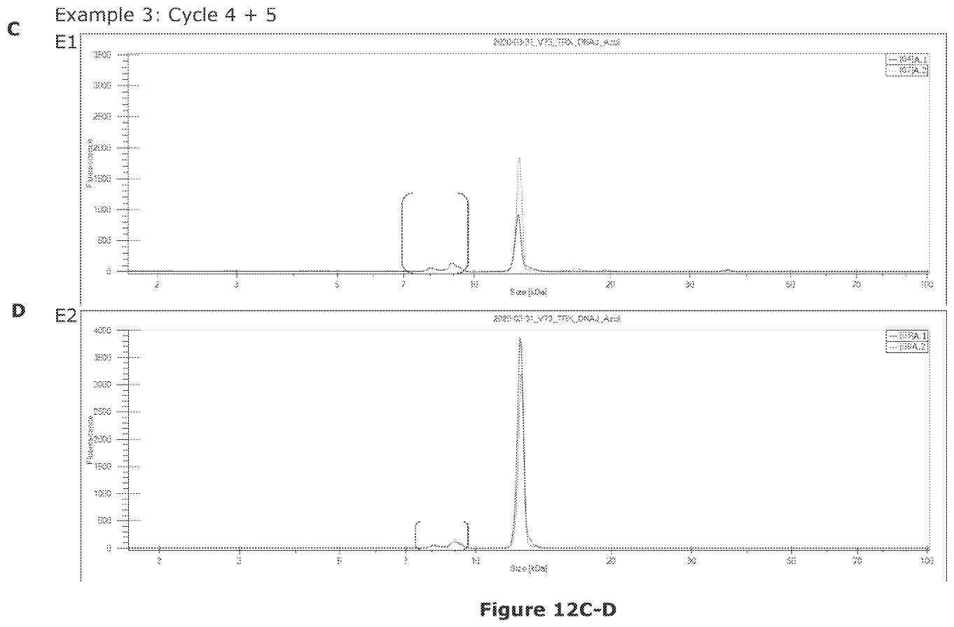

The purity was calculated from the elution fractions using a LabChip®GXII™ microfluidic electrophoretic separation system in combination with a Protein Express Assay Reagent Kit and a LabChip® HT Protein Express Chip. The calculated purity of the eluted target is consistent above 70.81%. Recorded purities are listed in Table 6. The overlay in FIG. 12 show the purity of all elution fractions.

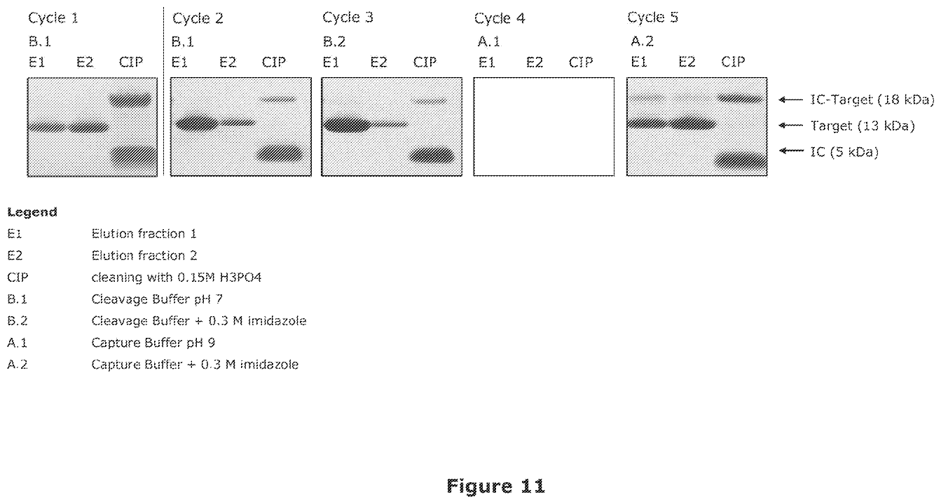

A sample of the elution (E1, E2) was analyzed by SDS-PAGE gel electrophoresis as shown in FIG. 11.

Table 6 shows results of the elution cycle study with an intein-N resin prototype column. The percentage of eluted target during five consecutive cycles of intein purification was calculated using the total protein amount recovered from elution (E1+E2) and the regeneration fractions (CIP: remaining intein-C target and intein-C fragment (IC)). The amount of eluted target was compared to the elution under standard conditions with (cycle 1 and 2) or without (cycle 4) introducing a pH shift as well as with 5 (cycle 1) or 10 (cycle 2-5) minutes residence time during the elution. By changing the flow rate, 10.81% more target yield could be recovered during the first elution step (E1). An additional enrichment of the elution buffer with imidazole increased the value to 12.51% higher yield during the first elution step. A 36.88% higher yield was recovered using imidazole containing elution buffer (A.2) during the first elution step (E1) of purification cycle 5. All fractions besides E2-fraction of cycle 3 contained nearly 70-90% pure tagless target.

TABLE 6

| Purification Cycle | 1 | 2 | 3 | 4 | 5 |
|---|---|---|---|---|---|
| Flow Rate at the Elution | 0.2 | 0.1 | 0.1 | 0.1 | 0.1 |
| | ml/min | ml/min | ml/min | ml/min | ml/min |
| Elution Buffer | Cleavage | Cleavage | Cleavage | Capture | Capture |
| | B.1 | B.1 | B.2 | A.1 | A.2 |
| Target | 13 kDa | 13 kDa | 13 kDa | 13 kDa | 13 kDa |
| Ligand | R46- | R46- | R46- | R46- | R46- |
| | 358132 | 358132 | 358132 | 358132 | 358132 |
| Total bound intein-C target/mg | 0.56 | 0.90 | 0.93 | 0.61 | 0.67 |
| E1/mg | 0.43 | 0.81 | 0.86 | 0.10 | 0.36 |
| E2/mg | 0.10 | 0.07 | 0.05 | 0.31 | 0.24 |
| E1 + E2/mg | 0.53 | 0.88 | 0.91 | 0.40 | 0.60 |
| Yield of cleaved target per total | 94.80% | 97.79% | 98.03% | 66.02% | 89.33% |
| bound IC-target | | | | | |
| % improvement in yield (total) | — | 2.99% | 3.23% | | 23.31% |
| Yield of cleaved target in E1 per total | 81.57% | 92.38% | 94.08% | 23.76% | 60.64% |
| cleaved IC-target | | | | | |
| % improvement in yield (E1) | — | 10.81% | 12.51% | | 36.88% |
| E1 Purity/% | 96.98 | 100.00 | 97.87 | 82.56 | 93.17 |
| E2 Purity/% | 98.77 | 93.43 | 70.81 | 96.94 | 90.67 |

### Example 5: Time Dependent Intein Cleavage Process Using Different Residence Times During the Elution of an Intein Column Purification

In this example, the flow rate of the intein purification method was set to 0.1 ml/min The time dependent optimization of the intein cleavage process according to longer residence times during the elution phase was investigated.

This example demonstrates the catalyzing effects that are triggered by the addition of additives like imidazole are more substantial to intein purification processes under fast flow rates (shorter residence times) (Example 2). The example further shows that the addition of such catalyzing agents increases the target elution yield and accelerates the reaction while keeping the pH constant between, e.g., the loading and the elution phase (Example 4). The effect in general could be verified for at least two different intein-C tagged target molecules.

The intein-N resin prototype column, carrying a third generation of intein-N ligand (R44-358132) was used in four consecutive cycles, but with a varied flow rate during the elution phase. One cycle of intein purification was accomplished with a 1 ml/min flow rate within all process steps (cycle 4). A combination approach of two different flow rates was investigated in purification cycle 1-3 using a flow rate of 1 ml/min flow for the equilibration, sample load, wash and CIP phase and a flow rate of 0.1/0.2 and 0.5 ml/min for the elution (cycle 1/2/3).

For all consecutive purification cycles, a pre-purified 13 kDa intein-C tagged target (using Strep-Tag® purification) was prepared and diluted to a concentration of 1 mg/mL. The intein-N resin prototype column was equilibrated with 5 CV Capture Buffer (100 mM Tris, 200 mM NaCl, pH 9) and the pre-purified intein-C target was loaded with 5 CV to the prototype column. The loading was accomplished under capture conditions (100 mM Tris, 200 mM NaCl, pH 9). The impurities were washed out with 5 CV Capture Buffer (100 mM Tris, 200 mM NaCl, pH 9). The elution of the 13 kDa target molecule was activated by a pH reduction step to a lower pH level using the Cleavage Buffer (100 mM Tris, 200 mM NaCl, pH 7). The elutions of the four cycles were performed in a two-step approach. The first elution was triggered under dynamic flow with 5 CV Cleavage Buffer. The second elution was triggered with 5 CV Cleavage Buffer after putting the flow of the column on hold to achieve a 2 h static column incubation. The chromatography column was regenerated using 10 CV acidic solutions with pH between 1-2 containing for example 0.15 M H₃PO₄ and was reused for the next round of intein purification as described above.

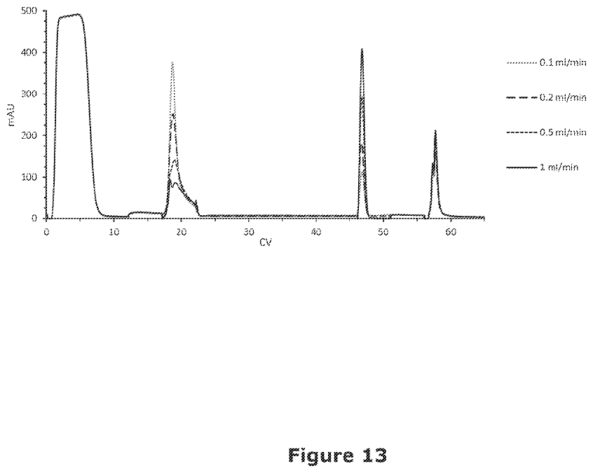

The overlay of cycle 1, 2, 3 and 4 (FIG. 13) demonstrates the different elution behaviour when varied residence time during the elution steps.

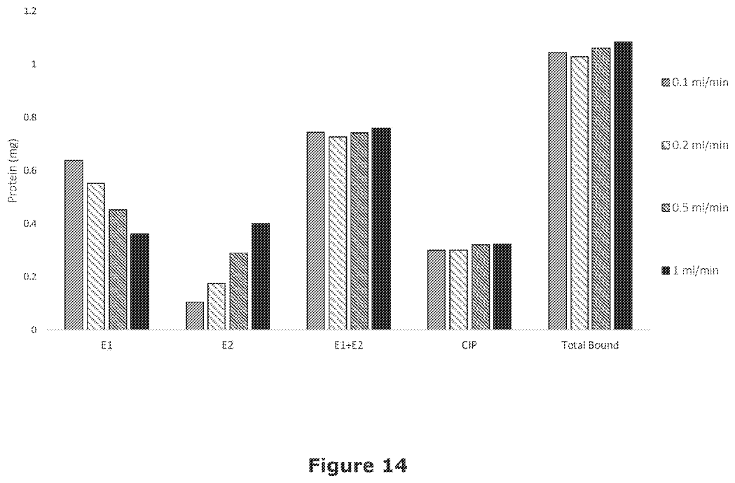

The amount of total bound protein as well as the protein amount recovered from the elution and CIP phases in all purification cycles is listed in Table 7 and the recovered protein amount is demonstrated in FIG. 14. Table 7 includes protein amount recovered from four intein purification cycles using different elution flow rates. The relative protein amount was recovered from the elution and CIP phases in relation to the total amount of bound protein.

TABLE 7

| Purification Cycle | 1 | 2 | 3 | 4 |
|---|---|---|---|---|
| Process Flow Rate | 1 | 1 | 1 | 1 |
| | ml/min | ml/min | ml/min | ml/min |
| Flow Rate during Elution | 0.1 | 0.2 | 0.5 | 1 |
| | ml/min | ml/min | ml/min | ml/min |
| Target | 13 kDa | 13 kDa | 13 kDa | 13 kDa |
| Ligand | R44- | R44- | R44- | R44- |
| | 358132 | 358132 | 358132 | 358132 |
| Total bound intein-C | 1.04 | 1.03 | 1.06 | 1.08 |
| target/mg | | | | |
| E1/mg | 0.64 | 0.55 | 0.45 | 0.36 |
| E2/mg | 0.11 | 0.18 | 0.29 | 0.4 |
| E1 + E2/mg | 0.74 | 0.73 | 0.74 | 0.76 |
| Yield of cleaved target | 71.15% | 70.87% | 69.81% | 70.37% |
| per total bound IC-target | | | | |
| % improvement in yield | 0.80% | 0.50% | −0.60% | — |
| (total) | | | | |
| Yield of cleaved target | 86.49% | 75.34% | 60.81% | 47.37% |
| in E1 per total cleaved | | | | |
| IC-target | | | | |
| % improvement in | 38.30% | 28.40% | 13.40% | — |
| yield (E1) | | | | |

The calculated amount of total bound protein remained almost constant at 1.04-1.08 mg. The accumulated protein amount recovered from the elution phases during all cycles remained constant at 0.74-0.76 mg implying about 70.37%-71.15% cleaved product compared to the total bound intein-C tagged target on the column. Only 47.37% of the total amount of recovered protein (yield) from the elution phases were recovered from the first elution phase of the reference run (cycle 4) using a constant flow rate at 1 ml/min for all process steps. With a lower flow rate of 0.5 ml/min or 0.2 ml/min, 60.81% or 75.34% yield was recovered from the first elution step. A reduction of the flow rate from 0.2 ml/min to 0.1 ml/min increased the relative amount of protein recovered from the first elution step up to 86.49%.

Whereas the previous examples demonstrated protein yield increase by changing the process flow rate from 0.2 ml/min to a combinatoric flow rate of 1 ml/min and 0.1 ml/min (Example 4, Cycle 1 and 2), this example verified the increased cleavage effect during the elution phase 1 that could be observed with an increased residence time, implying a smaller flow rate (0.5/0.2/0.1 ml/min) only during the elution. The elution amount that was collected during the elution step 1 was increased by 38.30% (cycle 1 compared to cycle 4).

### Example 6

Increased Intein Cleavage at Standard Cleavage Conditions (pH 7) Using Imidazole, Tested with Two Highly Glycosylated Target Molecules.

The exemplary intein-N resin prototype columns, carrying both a third generation of intein-N ligand (R46-358132 and R49-358132), were used each for two intein purification cycles (cycle 1 and 2 as well as cycle 3 and 4). The improvement of intein functionality using an imidazole-enriched cleavage condition buffer system (pH 7) was evaluated for the purification of two highly glycosylated target molecules.

The first two purification cycles were done with an intein-C tagged target molecule described as hEPO (modified from the corresponding UniProtKB—P01588 (EPO_HUMAN), MW=19 kDa). The intein-C tag was fused together with a signal binding peptide to the hEPO sequence and the fusion protein (28 kDa) was expressed in the CHOZN® GS−/− cell line and prepared as described above. The third and fourth purification run was conducted using the intein-C tagged receptor binding domain (RBD) of the S1 spike glycoprotein of SARS-CoV-2 described as S1-RBD (corresponding to the UniProtKB—PODTC2 (SPIKE_SARS2), MW=141 kDa, whereas the RBD has a MW of 26 kDa). The intein-C tagged S1-RBD including a mammalian signal peptide (33 kDa) was expressed using HEK293 cells. In all purification runs, the clarified mammalian cell supernatant (preparation described in Example 1), containing the secreted and processed intein-C tagged target molecules (hEPO and S1-RBD with a size of 24 kDa and 31 kDa) was used within this example. The stock of the molecules was adjusted to 0.1-0.3 mg/ml and pH 9.

The intein-N immobilized resins were equilibrated with 10 CV Capture Buffer (100 mM Tris, 200 mM NaCl, pH 9) and one of the above-referenced intein-C tagged target containing clarified mammalian cell supernatant was loaded with 5 CV to one of the intein-N resin prototype columns under capture conditions (100 mM Tris, 200 mM NaCl, pH 9). The unbound proteins were washed with 10 CV Capture Buffer (100 mM Tris, 200 mM NaCl, pH 9). The standard Cleavage Buffer described in this example as B.1 (100 mM Tris, 200 mM NaCl, pH 7) was enriched with 0.3 M imidazole (B.2). The intein cleavage reaction and tagless target release was triggered by a pH shift step to a lower pH value using one of the given Cleavage Buffers B.1 (cycle 1 and 3) or B.2 (cycle 2 and 4) and the elution was accomplished in a two-step approach. The first elution was triggered under dynamic flow with 6 CV Cleavage Buffer B.1 or B.2. The second elution was triggered with 4 CV Cleavage Buffer B.1 or B.2 after setting the flow on hold to achieve a 2 h static column incubation. The chromatography column was regenerated using at least 5 CV acidic solutions with pH between 1-2 containing for example 0.15 M H₃PO₄ and was reused for the next cycle of intein purification.

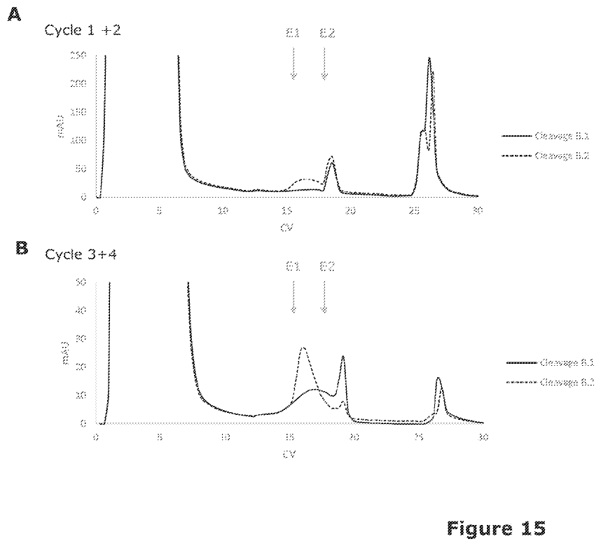

The A280-Absorbance chromatograms were analyzed according to the protein amounts in the elution and CIP fractions using the appropriate extinction coefficient of the targets. Two cutout overlays of the chromatograms demonstrate the different elution behavior during cycle 1 and 2 and the elution of the 19 kDa-target molecules (FIG. 15A) as well as during cycle 3 and 4 and the elution of the 26 kDa target molecule (FIG. 15B). The calculated protein amount that was collected during the elution 1, 2 and the CIP step is listed in Table 8. The amount of elution of the hEPO target was about 0.46-0.69 mg and 0.23-0.25 mg for the S1-RBP target, dependent of the used Cleavage Buffer B.1 or B.2. The total intein-C bound protein was calculated using the eluted target and the remaining intein-C target, that was released during the cleaning phase together with the remained intein-C fragments. The yield of tagless released target during the elution phase was calculated per total bound intein-C target. The yield was calculated to be 32.89% (cycle 1) and 80.54% (cycle 3) for the two targets using the standard Cleavage Buffer B.1. In comparison, 45.11% (cycle 2) and 82.49% (cycle 4) yield was achieved using the Cleavage Buffer B.2 (Table 8).

Thus, using both prototype columns and both target molecules, the yield as well as the amount of eluted target only during the elution phase 1 was increased using imidazole-enriched buffer (5.87% and 15.39% more protein yield during E1), demonstrating that the positive effect of azole- and azole-like structures on the intein cleavage kinetic and performance is applicable to highly glycosylated proteins.

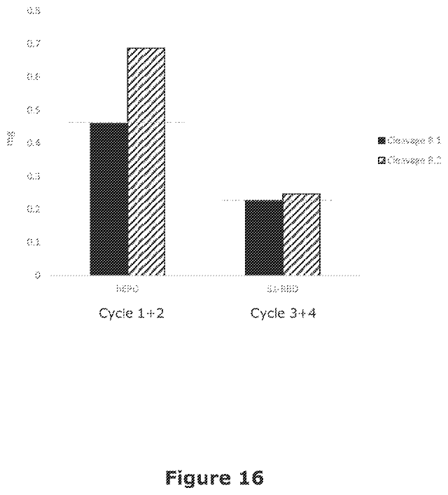

Table 8 shows the results of a cycle study with two intein-N resin prototype columns The percentage of eluted target during 4 consecutive cycles of intein purification was calculated using the total protein amount recovered from elution (E1+E2) and the regeneration fractions (CIP: remaining intein-C target and intein-C fragment (IC)). The amount of eluted target was compared to the elution under standard conditions (B.1). Thus, a 12.22% and 1.95% higher yield of cleaved target was recovered using imidazole containing elution buffer (B.2) during purification cycle 2 and 4, considering both elution steps. A 5.87% and 15.39% higher target amount could be recovered during the first elution step (E1) of cycle 2 and 4. The results are depicted in FIG. 16.

TABLE 8

| Purification Cycle | 1 | 2 | 3 | 4 |
|---|---|---|---|---|
| Elution Buffer | Cleavage | Cleavage | Cleavage | Cleavage |
| | B.1 | B.2 | B.1 | B.2 |
| Target | 19 kDa | 19 kDa | 26 kDa | 26 kDa |
| Ligand | R46- | R46- | R49- | R49- |
| | 358132 | 358132 | 358132 | 358132 |
| Total bound intein-C | 1.40 | 1.52 | 0.32 | 0.34 |
| target/mg | | | | |
| E1/mg | 0.25 | 0.41 | 0.18 | 0.24 |
| E2/mg | 0.21 | 0.28 | 0.08 | 0.04 |
| E1 + E2/mg | 0.46 | 0.69 | 0.26 | 0.28 |
| Yield of cleaved target | 32.89% | 45.11% | 80.54% | 82.49% |
| per total bound IC-target | | | | |
| % improvement in yield | | 12.22% | | 1.95% |
| (total) | | | | |
| Yield of cleaved target | 53.45% | 59.32% | 69.12% | 84.51% |
| in E1 per total | | | | |
| cleaved IC-target | | | | |
| % improvement in | — | 5.87% | — | 15.39% |
| yield (E1) | | | | |

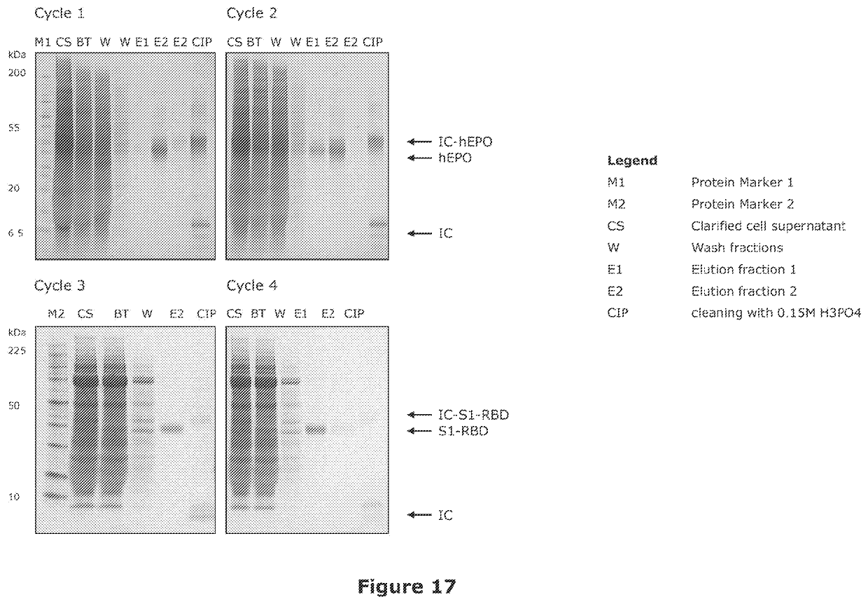

A sample size of selected fractions were analyzed by SDS-PAGE gel electrophoresis as shown in FIG. 17. An almost pure band in the elution fractions 1 and 2 of all purification runs was observed on the gel. This implies a high purity level of tagless released target during the elution. Of note, no sharp band was detected in the elution fractions, likely because of the different glycan patterns of the two targets. First, the glycosylation modifies the target molecules in several ways and second, glycosylation changes the size of the protein and thereby the running behavior on the SDS-Page, leading to a higher size on the SDS-Page than expected. Due to overloading effects of the column, some uncleaved intein-C target was observed as well.

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments, but should be defined only in accordance with the following claims and their equivalents.

1. A method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of:
(a) contacting the intein-C tagged target molecule with the intein-N polypeptide, for a first time period sufficient to form the intein complex; and
(b) releasing the target molecule from the intein complex by:
(i) contacting the intein complex with a medium effective to remove the target molecule, for a second time period which is longer than the first time period; and/or
(ii) contacting the intein complex with a heteroaromatic compound comprising at least one ring nitrogen atom.

2. The method according to claim 1, wherein the intein complex is formed during an intein-mediated process selected from the group consisting of protein purification, protein ligation, in vivo protein tagging, protein labelling, protein cyclization, protein polymerization, intein-induced reporter pathway analysis, and preparation of fusion proteins.

3. The method according to claim 1, wherein step (b) is performed at a pH that is lower than step (a).

4. The method according to claim 1, wherein steps (a) and (b) are performed at about the same pH.

5. The method according to claim 1, wherein the heteroaromatic compound is an azole or azole-containing compound.

6. The method according to claim 1, wherein the heteroaromatic compound is selected from the group consisting of an unsubstituted or substituted imidazole, pyrazole, 1,2,3-triazole, 1,2,4-triazole, tetrazole, pentazole, oxazole, isoxazole, 1,2,3-oxadiazole, 1,2,4-oxadiazole, furzan (1,2,5-oxadiazole), 1,3,4-oxadiazole, thiazole, isothiazole, thiadiazole (1,2,3-tiadiazole), 1,2,4-thiadaizole 1,2,5-thiadiazole, 1,3,4-thiadiazole, histidine, pyridine, pyrazine, pyrrole, pyrimidine, pyridazine, and any combination thereof.

7. The method according to claim 1, for purifying a target molecule, the method comprising the step of.
(a) contacting the intein-C tagged target molecule with the intein-N polypeptide on a chromatography resin at a first flow rate so as to form the intein complex; and
(b) releasing the target molecule from the intein complex at a second flow rate which is slower than the first flow rate.

8. The method according to claim 1, for purifying a target molecule, the method comprising the steps of
(a) providing a sample containing the intein-C tagged target molecule;
(b) loading the sample on a column comprising a chromatography resin, the chromatography resin comprising a covalently-linked N-terminal intein polypeptide, under conditions in which the intein-C polypeptide in the fusion protein binds to the intein-N polypeptide in the resin to form an intein complex, wherein the sample is loaded at a first flow rate/column residence time;
(c) optionally washing the resin containing the intein complex to remove unbound contaminants;
(d) cleaving the target molecule from the intein complex by:
(i) contacting the intein complex with a medium effective to cleave the target molecule at a second flow rate that is slower than the first flow rate, or a second column residence time that is longer than the first column residence time; and/or
(ii) contacting the intein complex with a heteroaromatic compound comprising at least one ring nitrogen atom;
(e) regenerating the chromatography resin;
(f) optionally, performing at least one additional purification cycle by repeating steps (a) to (e) at least once; and
(g) optionally, isolating the target molecule.

9. The method according to claim 8, wherein step (d) is performed at a lower pH than step (b) and optional step (c),
preferably, wherein step (b) comprises loading the intein-C tagged target molecule in a saline buffer having a pH of about 8 to about 10, more preferably a pH of about 9; and step (d) comprises contacting the intein complex with a saline buffer having a pH of about 6 to about 8, more preferably a pH of about 7.

10. The method according to claim 8, wherein step (d) is performed at about the same pH as step (b) and optional step (c);
preferably wherein steps (b), optional step (c) and step (d) are each performed in a saline buffer having a pH of about 8 to about 10, and more preferably,
wherein steps (b), optional step (c) and step (d) are each performed at a pH of about 9.

11. The method according to claim 8, wherein each of steps (b), (c) (if performed), (d) and (e) is independently performed under static incubation or constant flow representing residence times of 0.1-120 min per Column Volume (CV).

12. The method according to claim 8, wherein step (b) comprises contacting the chromatography resin with a cell culture supernatant comprising the intein-C tagged target molecule.

13. The method according to claim 8, wherein step (c) is performed, and comprises washing the chromatography resin with a washing buffer prior to releasing the target molecule from the intein-C polypeptide; preferably wherein the washing buffer comprises a detergent, a salt, a chaotropic agent, preferably urea or arginine, or a combination thereof.

14. The method according to claim 7, wherein the intein-N polypeptide is attached to the chromatography resin through a functional group selected from the group consisting of hydroxyl, thiol, epoxide, amino, carbonyl epoxide and carboxylic acid.

15. The method according to claim 7, wherein the second flow rate is at least about 2 times slower than the first flow rate, preferably between about 2 to about 20 times slower, more preferably between about 2 to about 10 times slower, and most preferably between about 5 to about 10 times slower;
or wherein the second column residence time is at least about 2 times longer than the first column residence time, preferably between about 2 to about 20 times longer, more preferably between about 2 to about 10 times longer, and most preferably between about 5 to about 10 times longer.

16. The method according to claim 1,
wherein the increase in contact time, the presence of the heteroaromatic compound and/or the reduction in pH during the target molecule release step increases the total yield of the target molecule by at least about 1%, or by at least about 2%, or by at least about 3%, or by at least about 5%, or by at least about 10%, or by at least about 15%, or by at least about 20%, or by at least about 25%, or
wherein the increase in contact time, the presence of the heteroaromatic compound and/or the reduction in pH during the target molecule release step increases the yield of the target molecule collected during elution step 1 (E1) by at least about 1%, or by at least about 2%, or by at least about 3%, or by at least about 5%, or by at least about 10%, or by at least about 15%, or by at least about 20%, or by at least about 25%, or by at least about 30%, or by at least about 35%, or by at least about 40%.

17. The method according to claim 1, wherein the target molecule is a protein, preferably wherein the sample is a crude protein preparation.

18. A method for releasing a target molecule from an intein complex comprising (i) a fusion protein comprising an intein-C polypeptide joined to the target molecule by a peptide bond (intein-C tagged target molecule); and (ii) an intein-N polypeptide, the method comprising the steps of contacting the intein complex with a heteroaromatic compound comprising at least one ring nitrogen atom.
